(19) United States
(12) Patent Application Publication
(10) Pub. No.: US 2021/0048429 A1
(43) Pub. Date: Feb. 18, 2021

(54) PLZF+ REGULATORY CD8 T CELLS FOR CONTROL OF INFLAMMATION

(71) Applicants: Vipin Kumar CHATURVEDI, San Diego, CA (US); THE REGENTS OF THE UNIVERSITY OF CALIFORNIA, Oakland, CA (US)

(72) Inventor: Vipin Kumar Chaturvedi, San Diego, CA (US)

(21) Appl. No.: 16/758,348

(22) PCT Filed: Oct. 23, 2018
(86) PCT No.: PCT/US2018/057112

§ 371 Date: Apr. 22, 2020

Related U.S. Application Data
(60) Provisional application No. 62/576,493, filed on Oct. 24, 2017.
(60) Provisional application No. 62/575,714, filed on Oct. 23, 2017.

(51) Int. Cl. G01N 33/50; C12N 5/0783; C07K 16/28

(52) U.S. Cl. CPC G01N 33/505; C12N 5/0637; C07K 16/2815; C12N 2501/2315; C07K 16/2803; C12N 2501/2302; C07K 16/2851

(57) ABSTRACT

Isolated immune cells having TCRαβ+, CD8αα+, Nk1.1+, PLZF+, CD161+, and optionally having one or more of CD11c+, CD137+ CD244+ or one or more of NK-inhibitory receptors, and methods of obtaining and using those cells, as well as methods to inhibit or enhance those cells in a mammal are provided.

### CROSS-REFERENCE TO RELATED APPLICATIONS

This application claims the benefit of the filing date of U.S. application Ser. No. 62/575,714, filed on Oct. 23, 2017, and U.S. application Ser. No. 62/576,493, filed on Oct. 24, 2017, the disclosures of which are incorporated by reference herein.

### BACKGROUND

The understanding of the process of immune tolerance is crucial for preventing autoimmunity as well as for the generation of effective cancer immunity. Conventional MHC-restricted TCRαβ⁺ T cells are involved in the development of several autoimmune disorders. A detailed knowledge of the cellular and molecular mechanism(s) controlling pathogenic self-reactive T cells is important for treatment of autoimmune diseases. T cells are controlled by both T cell-intrinsic and extrinsic cell-based mechanisms that prevent them from causing excessive tissue damage: immune ignorance, anergy, exhaustion, phenotype skewing and antigen-induced apoptosis are part of the intrinsic mechanisms whereas regulatory T cells (Treg), including Foxp3⁺CD4⁺ and Tr1, natural killer (NKT) cells and CD8⁺ T cells comprise cell-based mechanisms.

Since 1970s, it has been suggested that CD8⁺ T cells play an important role in immune regulation of autoimmune diseases, transplant tolerance and in homeostasis of cellular and humoral immune responses. Initially, using mice genetically deficient in or depleted of CD8⁺ T cells by treatment with anti-CD8 mAb, an important role for CD8 T cells in regulation of ENE and arthritis has been shown. Similarly, a critical regulatory role of CD122⁺CD8⁺ T cells involving a cytolytic mechanism has been shown in IL-2−/− and IL-2Rβ−/− mice. Interestingly, IL-2/IL-15Rβ-deficiency in humans also leads to a severe combined immunodeficiency syndrome as observed for IL-2Rα. Also, CD122⁺CD8⁺ T cells can provide barriers to stem cell engraftment, indicating the clinical relevance of CD8 Treg in humans. A critical role for CD8⁺ T cells in IL-2−/− mice lacking CD8⁺ T cells was shown as these animals develop colitis with an accelerated kinetics. CD8⁺ T cells also have been implicated in various conditions in humans, e.g. transplant survival, prevention of inflammatory bowel disease, and the treatment of multiple sclerosis with either glatiramer acetate (GA) or vaccination with irradiated, myelin basic protein-activated CD4⁺ T cells. Recently, GA-induced CD8⁺ Treg have been shown to eliminate CD4⁺ T cells in a HLA-E-restricted manner. In a recent clinical trial with anti-CD3 mAb (teplizumab), increased frequency of memory CD8⁺ T cells with regulatory gene expression was found to be associated with a positive clinical response in type I diabetes. Despite their critical role in control of autoimmunity, the biology of CD8⁺ Treg has not sufficiently advanced because of our inability to distinguish them from conventional CD8⁺ T cells based on either the cell surface phenotype or a specific transcription program similar to that described for Foxp3⁺CD4⁺ Treg.

CD8⁺ T regulatory cells have been a subject of study for many years, but this cell population has been difficult to define and identity. These immunosuppressive cells can be very important in the initiation and/or the maintenance of diseases when their numbers are skewed (high or low), such as in cancer, IBD, lupus, rheumatoid arthritis and various autoimmune diseases. The hardest part so far has been to identify and target these cells efficiently to modulate disease.

### SUMMARY

An immunosuppressor type of unconventional CD8⁺ T regulatory cell, which is more prevalent in the liver and in the gut, is identified. Adoptive transfer of these CD8⁺ T regulatory cells protect mice from developing antigen-induced EAE (a model for multiple sclerosis) and also protect mice from developing T cell-induced-colitis (a model for IBD). In other experiments, administration of anti-4-IBB antibody increases the number of these CD8⁺ T regulatory in the liver. Not only that, when the anti-4-1BB antibody was administered in mice with antigen-induce EAE, these mice show lesser symptoms than the negative controls. Similar results were obtained when anti-CD3 was administered in these EAE mice. Thus, innate-like PLZF⁺CD8αα Treg enriched in liver and in intestine play an important role in the homeostasis of immunity.

The disclosure provides for the use of antibodies to identify a CD8⁺ T regulatory cell population (e.g., by flow cytometry) in a subject with the following minimal phenotype: TCRαβ⁺ CD8αα⁺NK1.1⁺PLZF⁺ CD161⁺ in humans, CD11c⁺, though other markers are usually present too, for example one or more of CD137⁺ CD244⁺, or one or more of the NK-inhibitory receptors.

The disclosure provides for a method of treating cancer patients by administering monoclonal antibodies that bind to this CD8+ T reg cell population with the goal of lowering the number of this T cell population or eliminating these altogether in patients, allowing them to mount a proper immune response to the tumor.

The disclosure provides for a method of treating IBD, lupus, RA patients and patients suffering from other autoimmune diseases by administering Qa-1/HLA-E binding peptides that will stimulate CD8⁺T regs.

The disclosure provides for a method of treating IBD, lupus, RA patients and patients suffering from other autoimmune diseases by administering anti-CD3 in patients.

The disclosure provides for a method of treating IBD, lupus, RA patients and patients suffering from other autoimmune diseases by administering anti-CD137 (also known as anti-4-1BB).

The disclosure provides for sorting and expanding patient cells with cytokines IL-2/IL-15 and a combination of antibodies mentioned above.

In one embodiment, the cells are identified using monoclonal antibodies specific for CD8αα, NK1.1, PLZF, or CD161, and optionally antibodies specific for one or more of CD11c, CD137, CD244, TCRαβ, or one or more of NK-inhibitory receptors or a combination thereof. In one embodiment, the cells are human cells. In one embodiment, the method includes isolating the identified cells and optionally expanding the isolated cells. In one embodiment, the cells are from a patient with an autoimmune disease. In one embodiment, the cells are cultured with IL-2, IL-15, Qa-1/HLA-E binding peptides, anti-CD3 antibodies or anti-CD137 antibodies, or any combination thereof. In one embodiment, the composition is systemically administered. In one embodiment, the composition is locally administered.

Further provided is a method to prevent, inhibit or treat cancer in a mammal. The method includes administering to the mammal an effective amount of a composition comprising one or more antibodies specific for CD8αα, specific for NK1.1, specific for PLZF, or specific for CD161, or a combination thereof and optionally a composition comprising one or more antibodies specific for CD11c, specific for CD137, specific for CD244, specific for TCRαβ, or specific for one or more of NK-inhibitory receptors, or a combination thereof. In one embodiment, the cancer is neck cancer. In one embodiment, the cancer is melanoma. In one embodiment, the mammal is a human. In one embodiment, the composition is systemically administered. In one embodiment, the composition is locally administered.

A method to prevent, inhibit or treat autoimmune disease in a mammal is provided. The method includes administering to the mammal a composition comprising one or more Qa-1/HLA-E binding peptides, anti-CD3 antibodies or anti-CD137 antibodies in an amount effective to stimulate CD8α+T regs. In one embodiment, the disease is IBD, colitis, lupus or RA. In one embodiment, the mammal is a human. In one embodiment, the composition is systemically administered. In one embodiment, the composition is locally administered.

### BRIEF DESCRIPTION OF FIGURES

FIGS. 1A-H. Liver enriched CD8 Treg are PLZF positive and utilize an innate-like PLZF-driven transcription program. (A) Representative flow cytometry plots showing gate strategy for murine CD8 Treg in liver mononuclear cells (MNCs) from naïve B6 mice. CD8 Treg were identified as B220⁻CD4⁻TCRβ⁺CD8α⁺β⁻ cells. Scatter graphs show cumulative data of percentage and number of hepatic CD8 Treg among TCRβ⁺ T cells in naïve B6 mice (n=21). Each dot represents an individual mouse. Results shown as mean ±SEM. (B) Histograms show CD69, NK1.1, CD44 and CD62L expression by CD8 Treg (top) and CD8_{conv} (bottom) compared with isotypes (filled gray) as determined by flow cytometry. Numbers in histograms indicate the percentage of positive cells, (C) Representative Dot plots showing CD8 Treg and CD8_{conv} (top) as well as iNKT cells and CD4 T cells (bottom). Histograms show expression of PLZF or GFP (reporting PLZF) by CD8 Treg and CD8_{conv} (top) as well by iNKT cells (CD4⁺αGalCer/CD1d tetramer⁺) and CD4⁺ T cells (CD4⁺αGalCer/CD1d tetramer⁻) (bottom) from naïve B6 mice or PLZF-eGFP reporter (PEG) mice. CD8 Treg and iNKT cells (open); CD8_{conv} and CD4⁺ T cells (filled gray). (D) Quantitative RT-PCR analysis of PLZF expression on sorted CD8 Treg and CD8_{conv} following normalization using β actin gene. (E) Expression of TdTomato in CD8 Treg and CD8_{conv} from livers of B6 (top) and Pcre x R26T (26T) (bottom) mice. CD8 Treg and CD8_{conv} were stimulated for 3 days with anti-CD3/anti-CD28 mAbs and TdTomato expression was measured by FACS on sorted cells. Numbers indicate percentage. (F) Quantitative RT-PCR analysis of selected transcription factors expression on sorted CD8 Treg. The expression of each transcription factor is represented by mRNA fold change to that on CD8_{conv}. (G) Numbers of CD8 Treg in liver and spleen of homozygous PLZF−/− mice and littermates PLZF+/+ and PLZFH+/− mice as determined by FACS. Data represent mean ±2SD. *p≤0.05, **p≤0.01, unpaired t test. (H) Numbers of CD8 Treg in livers of homozygous RORα−/− or Id3−/− mice and B6 mice (RORα+/+ or Id3+/+) as determined by FACS. Data represent mean ±2SD. **p≤0.01, ***p≤0.001, unpaired t test. Data. representative of three independent experiments.

FIGS. 2A-I. Hepatic CD8 Treg are innate-like and a substantial portion of CD8 Treg co-express CD244 and CD11c. (A) Representative dot plot showing CD244 and CD11c expression by hepatic CD8 Treg (left) and CD8_{conv} (right) from naïve B6 mice following gate strategy. Numbers indicate % of CD8 Treg or CD8_{conv} that were CD244⁺ or CD244⁺CD11c⁺. (B) Bar graphs show percentage (right) and numbers (left) of CD244⁺CD11c⁺ CD8 Treg in liver MNCs from PLZF−/− mice (n=3) and littermates PLZF+/+ and PLZF+/− mice (n=4). Data are represented as mean ±2SD. **p≤0.01, unpaired t test. (C) Sorted hepatic CD8αα⁺CD11T cells (2×10⁵/mouse) from naïve B6 mice were adoptively transferred i.v. into naïve B6 mice one day prior to EAE induction. EAE was also induced in non-transferred naïve B6 mice (control, 3-5 mice). Mean disease scores ±SEM are shown on the y-axis versus days post-immunization on the x-axis. (D) Representative dot plot showing NK1 .1, NKG2D, CD137 (4-1BB) and CD200 expression by hepatic CD244⁺CD11c⁺ CD8 Treg from naïve B6 mice. (E) CD8 Treg are not Foxp3⁺ and represent a distinct population of Treg. Histograms show Foxp3 and GITR expression by CD8 Treg (black line) and CD8_{conv} (gray line) in liver MNCs from naïve B6 mice. (F) Histograms show CD25, CD122, PD-1, CD28, CD27, OX40, CD200 and 4-1BB expression by CD8 Treg (top) and CD8_{conv} (bottom) compared with isotypes (filled gray). Numbers indicate percentage of positive cells. (G) Histograms show ICOS, CXCR5, CD127, Eomes and CD103 expression by CD8 Treg (top) and CD8_{conv} (bottom) compared with isotypes (filled gray). Numbers indicate percentage of positive cells. (H) Histograms show the expression of NK cell surface markers, including Ly49A, Ly49E/F, Ly49G, Ly49G2, Ly49I, Ly49D, Ly49H and NKG2D, by CD8 Treg (top) or CD8_{conv} (bottom) compared with isotypes (filled gray), Numbers indicate percentage of positive cells. (I) Quantitative RT-PCR analysis of the expression of selected genes on sorted CD8 Treg from liver of naïve B6 mice. The expression of each gene is represented by mRNA fold change to that on CD8_{conv}. Data representative of three independent experiments.

FIGS. 3A-B. CD8 Treg are also present in other lymphoid compartments and originated in the thymus. (A) Percentage of CD8 Treg in bone marrow, thymus, liver, lung, spleen and peripheral blood of naïve B6 mice (n=3-4). Each dot represents data from an individual mouse. Horizontal line identified mean. (B) Percentage of CD8 Treg in liver MNCs from athymic mice (n=3) and euthymic heterozygous littermate (n=3). Data represented as mean ±SEM. Data representative of two independent experiments.

FIGS. 4A-G. Hepatic CD8 Treg have immune regulatory properties and are physiologically expanded during EAE. (A) Adoptive transfer of sorted CD8 Treg protects mice from MOG-induced EAE (a model for multiple sclerosis) Sorted hepatic CD8 Treg or CD8_{conv} (1×10⁵/mouse) from naïve B6 mice were adoptively transferred i.v. into groups of naïve B6 mice one day prior to EAE induction with MOG₃₅₋₅₅/CFA/PT. As a positive control, EAE was also induced in non-transferred naive B6 mice. EAE clinical severity (disease scores) was monitored daily in all groups. Mean disease scores ±SEM are shown on the y-axis versus days post-immunization on the x-axis. *p≤0.05, Student t test. Data representative of 3-5 mice analyzed in each group. (B) Sorted hepatic CD8 Treg or CD8_{conv} (2×10⁵/mouse,) from naïve CD1d−/− mice were adoptively transferred i.v. into groups of naïve B6 mice one day prior to EAE induction as described in A. Mean disease scores ±SEM are shown. *p≤0.05, Student t test. Data representative of 3-5 mice analyzed in each group. (C) CD8 Treg are increased during the recovery phase of EAE. EAE was induced in groups of naïve B6 mice with either MOG₃₅₋₅₅ or hen egg lysozyme (HEL) peptide along with CFA/PT and the clinical disease scores were evaluated daily. Recipient MOG₃₅₋₅₅-immunized B6 mice were sacrificed at day 10 (n=3, EAE d10) or day 25 (n=9, EAE d25) while HEL-immunized mice were sacrificed at day 25 (n=3, HEL d25). The percentage of hepatic CD8 Treg was determined by FACS and compared to that in naïve B6 mice (n=5). Data are represented as mean ±SEM. *p≤0.05, unpaired t test. (D) Percentage of CD8 Treg (y-axis) versus EAE clinical disease scores (x-axis) in spleens of MOG₃₅₋₅₅/CFA/PT-immunized B6 mice (n=16) at day 30 during the recovery phase of EAE are shown. Data are represented as mean ±SEM. **p≤0.01, unpaired t test. Data representative of three independent experiments. (E) Adoptive transfer of sorted CD8 Treg protects mice from CD45Rb^{high}CD4⁺ T cell-induced colitis (a model for IBD) in a perforin-dependent manner. Groups of Rag 1−/− mice (n=4) were reconstituted i.v. with sorted 4×10⁵ CD4⁺CD25⁻CD45RB^{high} T cells from spleen of naïve B6 mice alone (control) or co-transfer with 2×10⁵ sorted CD8 Treg from liver of naïve B6 (perforin+/+) or perforin−/− mice. Body weight change was monitored weekly for 4 weeks and expressed as ratio of the weight before cell transfer. Representative colon histopathology examination by ME staining in the indicated group of Rag1−/− recipients 4 weeks after cell transfer. Magnifications: 100×. Results are representative of two independent experiments. (F) Numbers of CFSE-labeled OT-II CD4⁺ T-cells recovered from B6 recipients following co-transfer of OT-II CD4⁺ T cells (1×10⁶) with either positive selected CD8⁺ T cells or liver MNCs (8×10⁶ cells/mouse) from B6 (perf+/+) or perforin−/− mice. Transferred B6 mice were injected with OVA₃₂₃₋₃₃₉ peptide (50 μg/mouse, ip) 24 hours after adoptive transfer. As control, transferred B6 mice were injected with either PBS or OVA peptide. After 3 days, splenocytes were analyzed for expression of CFSE-labeled OT-II CD4⁺ T-cells by FACS. Results represented as mean ±SEM. *p≤0.5, **p≤0.01, one way ANOVA with Tukey's multiple comparisons test. (G) Percentage of CFSE-labeled CD4⁺ T cells recovered following in vitro stimulation. CFSE-labeled sorted CD4⁺ T cells from spleen of B6 mice were cultured alone or co-cultured with either sorted CD8 Treg or CD8_{conv} followed by in vitro stimulation with plate-bound anti-CD3 mAb (100 μg/well) and irradiated APC. After 4 days, CFSE-labeled cells were analyzed by FACS. Results represented as mean ±SEM. **p≤0.01, t test.

FIGS. 5A-E. Hepatic CD8 Treg secrete typical PLZF-driven cytokines, are dependent on IL-15/IL-2Rβ for their development and most of them are Qa-1^{b}-restricted. (A) Production of IFNγ, IL-2, TNFα, IL-4, IL-17A, IL-6 and IL-10 by CD8 Treg or CD8_{conv} (1×10⁵ cells/well) sorted from liver of B6 mice in response to in vitro stimulation with plate-bound anti-CD3 (1 μg/ml) mAb. Cytokine production was examined at various time points in supernatants from cultured cells by BD™ Cytometric Bead Array (CBA). Values are expressed in pg/ml. **p≤0.01, unpaired t test. (B) Percentage of CD8 Treg in liver MNCs from CD25−/−, CD122−/−, IL-15−/−, IL-7−/−, IL-6−/−, IFNγ−/− and T-bet−/− mice compared with B6 mice as determined by FACS. Each dot represents an individual mouse. Data representative of 3-4 mice per group. Data are represented as mean SEM. ***p≤0.001, unpaired t test. (C) Percentage of CD8 Treg in liver MNCs from CD8α−/−, CD8β−/−, μMT−/−, CD1d−/−, Jα18−/−, Qa-1^{b}−/− and TAP1−/− mice compared with 96 mice as determined by FACS. Each dot represents an individual mouse. Data representative of 3-5 mice per group. Data are represented as mean ±SEM. *p≤0.05, ***p≤0.001, ****p≤0.0001, unpaired t test. (D) Serum ALT levels from groups of B6 (n=6) and Qa-1−/− (n=5) sacrificed 24 hours after i.v. injection with ConA (170 μg/20 g body weight). **p≤0.01, unpaired t test. E. H&E staining of liver sections of B6 and Qa-1−/− mice following ConA injection as before. Magnification ×100. Data representative of three independent experiments.

FIGS. 6A-F. Hepatic CD8 Treg are polyclonal. (A) Bar graph showing TCR Vβ chain expression on hepatic CD8 Treg from naïve B6 mice (n=3) by FACS. Liver MNCs were stained with anti-TCRβ, anti-CD8α, anti-CD8β.2 and distinct anti-TCR Vβ mAbs. Data are represented as mean ±SEM. (B) Frequency distribution of TRBV gene segment usage by all unique clonotypes from sorted CD8 Treg (n=31, left) and CD8_{conv} (n=7256, right) isolated from liver MNCs from naïve B6 mice (n=10) and analyzed by high-throughput sequencing of the CDR3β regions. Each TRBV gene segment is represented by a slice proportional to its average frequency. (C) Frequency distribution of TRBJ gene usage by all unique clonotypes from sorted CD8 Treg (white bar) and CD8_{conv} (black bar). (D) CDR3 length distribution by all unique clonotypes from sorted CD8 Treg (white bar) and CD8_{conv} (black bar). (E) Bar graph showing TCR Vα chain expression on hepatic CD8 Treg from naïve B6 mice. Liver MNCs were stained with anti-TCRβ, anti-CD8α, anti-CD8β.2 and available anti-TCR Vα mAbs. (F) RT-PCR analysis of TCR Vα gene segments expression in CD8 Treg and CD8_{conv}. Total RNA was extracted from sorted CD8 Treg and CD8_{conv} from liver MNCs of naïve B6 mice. The TCR Vα chain was amplified. using specific primers for each Vα gene and a common Cα primer. The PCR products were electrophoresed in 1.2% agarose gel and visualized by ethidium bromide staining.

FIGS. 7A-B. CD8αα Treg are enriched in colonic IEL, and colonic IEL CD8 Treg express NK-inhibitory receptors. A. Contour plots represent live CD8⁺ T cells (gating: CD45⁺TCRβ⁺CD4⁻B220⁻) from different tissues in gut and Gut-associated lymphoid tissue (GALT) in WT B6 mice. Numbers indicate % of CD8 Treg (left) and CD8αβ T cells (tight). Histogram overlay shows intracellular PLZF expression in colonic CD8 Treg (red histogram) using an anti-PLZF mAb (left) or as GFP expression in PLZF-GFP mice (right). Blue histograms show control staining in non CD8αα, T cells (left) or in non-GFP mice (right). Plots are representative of at least 2 independent experiments for each tissue. B. CD45⁺TCRβ⁺CD8αα/αβ live cells from colonic IELs were gated for lymphocyte activation marker, CD44 and IL-2 receptor β chain, CD122. The expression of NK-inhibitory receptors was analyzed on CD44⁺CD122⁺ colonic IEL CD8 Treg and IEL CD8αβ T cells. Square gates within contour plots show percentage of Ly49 C/I/F/H⁺ cells in B6 (WT) colonic IEL CD8 Treg (first panel), WT colonic IEL CD8αβ T cells (second panel), Qa-1^{b}−/− colonic IEL CD8 Treg (third panel) and Batf3−/− colonic IEL CD8 Treg (fourd panel). Numbers indicate the frequency of events.

FIGS. 8A-C. Spontaneous inflammation in Qa-1^{b}−/− mice genetically deficient in CD8 Treg. A. Inflammation in liver of Qa-1^{b}−/− mice. Representative H&E and Sirius-red staining of liver sections from naïve Qa-1^{b}−/− mice. Magnification ×100. B. Infiltration of inflammatory CD4 T cells into liver of Qa-1^{b}−/− mice. Representative H&E, anti-CD4 and anti-CD8 staining of liver sections from inflamed livers of naïve Qa-1^{b}−/− mice. Magnification ×200. C. Spontaneous inflammation in colonic tissue in Qa-1^{b}−/− mice. H&E staining of colonic sections showing big patch of cellular infiltrates in Qa-1^{b}−/− mice (Magnification ×4). Bar graph shows IL-6 secretion from colon explant culture from B6 (WT) and Qt-1^{b}−/− mice as measured by ELISA.

FIGS. 9A-H. Activation/expansion of CD8 Treg by TCR-derived peptides protect mice from MOG-induced EAE as well as from DSS-induced colitis in a Qa-1-dependent fashion. A. Proliferative response of splenocytes from B6 (Qa-1^{b}+/+) (white bar) and Qa-1^{b}−/− (black bar) mice against in vitro stimulation with individual TCR-derived peptides. Proliferation was measured by incorporation of [³H]-thymidine in triplicate cultures. Bar graph depicts mean ±2SD of stimulation index. *p≤0.05, paired t test. B. Percentage of CFSE-labeled CD4+ and CD8α+ T cells in response to in vitro stimulation with individual TCR-derived peptides. Proliferation by CBE dilution analysis was examined in splenocytes of B6 mice. Gating was performed on CD4+ (white bar) and CD8α+ (gray bar) T cells. Data plotted are mean ±2SD. *p≤0.05, paired t test. C. EAE incidence in B6 mice after vaccination with specific TCR-derived peptides. Groups of B6 mice were vaccinated i.p. with 50 μg of one of the following peptides: p4L, p5L, p6L, p8.2L, p11L, p5C and p42-50 in IFA or with PBS in IFA as control. One week later, EAE was actively induced following immunization with MOG₃₅₋₅₅/CFA/PTx. The clinical symptoms of EAE were monitored and scored daily until day 30. Results represent the mean ±SD. *p≤0.05, Student t test. D. Cytokine response after vaccination with peptide p8.2L. B6 mice were vaccinated with either peptide p8.2L or control peptide p6L. One week after, EAE was induced by injection of MOG₃₅₋₅₅/CFA/PTx. On day 20, splenocytes from vaccinated mice were cultivated and challenged in vitro with MOG₃₅₋₅₅ (40 μg/ml) for 72 hours. Supernatants were collected and analyzed by CBA (BD). Results in graphs shown as mean ±SEM of pg/ml cytokine secretion. *p≤0.05, unpaired t test. E. CD8 Tree infiltrate into CNS during EAE. Representative flow cytometry dot plots showing percentage of CD8 Treg (left) and CD8αβ (right) T cells in liver (top) and CNS (bottom) of B6 mice following vaccination with peptide p8.2L on day 12 after EAE induction in comparison with control mice. Bar graphs showing percentage of CD8 Treg in liver (top) and CNS (bottom) of p8.2L-vaccinated B6 mice. **p≤0.01, unpaired t test. F. Bar graphs showing percentage of CD4+ and CD8+ T cells in liver and CNS of p8.2L-vaccinated B6 mice and control mice. G. (Left) EAE incidence in B6 (Qa-1^{b}+/+) and Qa-1^{b}−/− mice after vaccination with peptide 8.2L. B6 and Qa-1^{b}−/− mice were vaccinated i.p. with 50 μg of peptide 8.2L in IFA or PBS/IFA (control). One week later, EAE was induced with MOG₃₅₋₅₅/CFA/PTx. (Right). EAE incidence in SJL mice after vaccination with peptide 8.2L. SJL mice were vaccinated i.p. with 50 μg of peptide 8.2L in IFA or PBS/IFA (control). One week later, EAE was induced with PLP₁₃₉₋₁₅₁/CFA/PTx. All mice were monitored for disease symptoms until day 25. Results represent the mean SD. These data are representative of three independent experiments. H. Treatment with a CD8 Treg-inducing peptide confers protection from DSS-induced colitis in WT but not in Qa-1^{b}−/− mice, Groups of B6 (WT) (upper panels) or Qa-1^{b}−/− (lower panels) mice were supplied with 2.5% DSS containing water for 7 days following prophylactic (3 days prior) i.p. injection of the Qa-1^{b}-binding peptide p8.2L (50 μg/mouse) as indicated (+P) or control (−P). The relative reduction in colon length (gross morphology) as well as patches of cellular infiltrates in epithelial damage (white arrows) using H&E staining of colonic section are shown. Bar graphs (middle panels) indicate cumulative clinical scores in one representative experiment (each dot represents individual mouse). Cumulative clinical scores were calculated on the day of sacrifice (peak disease by body weight) by the following: i) grading loss in body weight (0 to 4% as 0; 4 to 10% as 1; 10 to 15% as 2; 15 to 20% as 3 and a loss of >20% as 4); ii) grading colon length decrease (0.5 to 1 cm as 1; 1.1 to 1.5 cm as 2; 1.6 to 2 cm as 3 and >2 cm as 4); iii) average colon thickness of proximal and distal colon in mm; iv) stool softness on a scale of 0 to 3 with 0 for regular hard stool and 3 for liquid stool. Bar graphs (right panels) show cytokine secretion in supernatants from colon explant culture (1 cm tissue from distal end) using CBA kit. MFI values for each pro-inflammatory cytokine were compared between peptide treated and untreated groups. *p<0.05, **p<0.01, ***=p<0.001 (unpaired t test).

FIG. 10. Requirement of migratory DC in CD8 Treg-mediated protection from DSS-induced colitis. Treatment with peptide p8.2L (50 μg/mouse) fails to prevent colitis in Batf3−/− mice deficient in migratory CD103⁺ DC but not in other DC populations. Age matched WT and Batf3−/− male mice were treated with peptide prophylactically and challenged with 2.5% DSS in drinking water. Gross photography (left) and cumulative clinical disease scores (right) are shown. Each dot in the bar graphs represents one mouse. ****p<0.0001, Student's t test.

FIGS. 11A-F. Agonistic anti-4-1BB Ab administration leads to expansion/activation of CD8 Treg and protection from EAE in a Qa-1 dependent manner. A. Groups of B6 mice were administered i.v. with 25 μg of 4-1BB Ab and 72 hr later BrdU⁺ CD8 Treg were quantified in liver as shown. B. In parallel, frequency of hepatic CD11c⁺2B4⁺ CD8 Treg was determined as shown. **p<0.01. C. Groups of B6 mice (n=5) were injected i.p. with either 25 μg or 200 μg/mouse of anti-4-1BB Ab or an isotype matched control Ab and the next day challenged with MOG₃₅₋₅₅/CFA/PTX for the induction of EAE. Clinical scores were recorded daily as shown. D. Groups of Qa-1−/− mice (n=4-5) were injected with 25 μg/mouse of anti-4-1BB Ab and immunized for the induction of EAE as in C. Clinical scores are shown. E. Reduced number of CD8 Treg and CD11c⁺2B4⁺ CD8 Treg in 4-1BB−/− mice genetically deficient in 4-1BB molecule. F. Induction of CD8 Treg following administration of anti-4-1BB Ab and subsequent protection from EAE.

FIGS. 12A-E. Induction of CD8 Treg following administration of anti-CD3 mAb and subsequent protection from EAE. A. Representative dot plots and cumulative data of CD8 Treg in spleen and liver of B6 mice after anti-CD3 mAb treatment. Female B6 mice were immunized with anti-CD3 mAb i.p. on day 1 and day 3. On day 7, mice were sacrificed and spleen and liver mononuclear cells were isolated and stained for FACS. B. The frequency of CD8 Treg in liver mononuclear cells was monitored daily by FACS after immunization with 200 μg of anti-CD3 mAb. C. Groups of female B6 mice were injected i,p. with 200 μg of anti-CD3 mAb and five days later challenged with MOG₃₅₋₅₅/CFA/PTX for the induction of EAE. Clinical scores were recorded daily as shown. *p<0.05, Student t test. D. Female B6 mice were immunized with anti-CD3 mAb i.p. on day 1 and day 3. On day 7, mice were sacrificed and spleen cells were isolated and stained with various fluorochrome labeled antibodies. CD8 Treg and CDαβ T cell proliferation was measured by BrdU incorporation. E. Anti-CD3 mAb is the best anti-T cell surface antibody for induction of CD8 Treg. Anti-CD3 (clone 2C11) mAb activates/expands CD8 Treg in liver. Naïve female B6 mice were administered i.p. with various antibodies (200 μg) or just with PBS (control). Five days later, liver mononuclear cells were isolated and stained with CD8α, CD8β and TCRβ fluorochrome labeled antibodies. H-157 (anti-TCRβ), GK1.5 (anti-CD4), 2.43 (anti-CD8), YTS177 and YTS 05 are also different anti-CD4 mAbs.

FIG. 13. Low frequency of CD8 Tregs in liver mononuclear cells from Yaa lupus mice. Representative dot plot showing hepatic CD8 Treg in two 10 weeks old BXSB-Yaa mice (right) and one age-matched B6 mouse (right). Numbers indicate % of CD8 Treg.

FIG. 14. Upregulation of several proinflammatory genes in liver of Qa-l^{b}−/− mice deficient in CD8 Treg. Volcano plot displaying each gene's -log10 (p-value) and log2 fold change with the selected covariate. Highly statistically significant genes fall at the top of the plot above the horizontal lines, and highly differentially expressed genes fall to either side. Horizontal lines indicate various False Discovery Rate (FDR) thresholds or p-value thresholds if there is no adjustment to the p-values. Genes are colored if the resulting p-value is below the given FDR or p-value threshold. The 40 most statistically significant genes are labeled in the plot.

FIG. 15. We have successfully generated several founders (mice) in which the allele of Zbtb16 (PLZF) containing loxp sites flank the exon that encodes the BTB domain and the zinc fingers. The place where the loxp oligos were inserted and the sequencing scans showing the successful insertion of the loxp sites are shown. These founders are currently being bred with CD4-Cre to generate mice lacking PLZF+ T cells, including CDS Treg.

FIGS. 16A-E. CD8 Treg are also present in human peripheral blood and express similar cell surface markers. A. Representative flow cytometry plots showing gating strategy for the identification of circulating CD8 Treg in human PBMC. The total CD8⁺ gate was identified using anti-CD8α and anti-CD8β mAbs within the TCRαβ⁺ gate; after intracellular staining for PLZF, CD8⁺PLZF⁺ T cells were gated within the CD8⁺ population; using anti-CD8α and anti-CD8β mAbs, CD8α⁺β⁻ T cells were identified within the CD8⁺PLZF⁺ gate; finally, based on expression of CD161 and the invariant TCR Vα7.2, CD8 Treg were defined as PLZF⁺TCRαβ⁺CD8αα⁺ T cells negative for TCR Vα7.2/Jα33 expression and with intermediate or low expression of CD161 (CD161^{+/-}) while MAIT cells were identified as CD161⁺⁺TCR Vα7.2/Jα33⁺. B. B. Cumulative data for the frequency of CD8 Treg in PBMC from healthy donors (n=25) following gate strategy. Each dot represents an individual donor. Results shown as mean±SEM. C. Histogram profile and cumulative data of Geo MFI of CD244, CD11c and Granzyme B expression by circulating CD8 Treg compared with both CD8_{conv} and MAIT cells. Each dot represents individual donors. Data were obtained from 13 to 16 healthy donors. Results shown as mean±SEM. *p≤0.5, **p≤0.01, ***p≤0.001, ****p≤0.0001, one-way ANOVA with Bonferroni's multiple comparisons test. D. (Top) Representative flow cytometry plots showing co-expression of CD244 and CD11c by circulating CD8 Treg and MAIT cells. Gate indicates CD244⁺CD11c⁺ cells and numbers indicate percentage of CD8 Treg or MAIT cells that are CD244⁺CD11c⁺. Scatter graph shows percentage of CD8 Treg or MAIT cells that are CD244⁺CD11c⁺ in PBMC from healthy donors (n=9). **p≤0.01, Wilcoxon matched-pairs signed rank test. (Bottom) Representative flow cytometry plots showing co-expression of Granzyme B and perforin gated on CD244⁺CD11c⁺ circulating CD8 Treg or MAIT cells. Numbers in dot plots show the percentage of events in each quadrant. Scatter graph shows percentage of CD244⁺CD11c⁺ CD8 Treg or MAIT cells that are Granzyme B⁺Perforin⁺ in PBMC from healthy donors (n=2). **p≤0.01, unpaired t test. E. Frequency distribution of TRBV gene segment usage by all unique clonotypes from sorted circulating CD8 Treg (n=794, left) and CD8_{conv} (n=12,244, right) isolated from PBMC of one healthy donor and analyzed by high-throughput sequencing of the CDR3β regions. Each TRBV gene segment is represented by a slice proportional to its average frequency. The CDR3 was defined as starting at the last cysteine encoded by the 3′ portion of the Vβ gene segment and ending at the phenylalanine in the conserved TRBJ segment motif FGXG.

FIGS. 17A-B. A. Cumulative data of Geo MFI of IFNβ, IL-17A, TNFα and IL-4 (top) as well as IL-18Rα1, RORγt, CXCR6 and CCR6 (bottom) expression by circulating CD8 Treg compared with both CD8_{conv} and MAIT cells. Each dot represents individual donors. Data were obtained from 6-12 healthy donors. Results shown as mean±SEM. *p≤0.5, **p≤0.01, ***p≤0.001, one-way ANOVA with Bonferroni's multiple comparisons test. B. Frequency distribution of TRBJ gene usage (top) and CDR3β lengths (bottom) by all unique clonotypes from sorted CD8 Treg (white bar) and CD8_{conv} (black bar) from human PBMC. Both CD8 Treg and CD8_{conv} were sorted from peripheral blood of one healthy donor and subjected to high-throughput sequencing of the CDR3β regions. The CDR3 was defined as starting at the last cysteine encoded by the 3′ portion of the Vβ gene segment and ending at the phenylalanine in the conserved TRBJ segment motif FGXG.

FIGS. 18A-E. Circulating CD8 Treg are increased in PBMCs from chronic Rheumatoid Arthritis (RA) patients. Cumulative data of (A) percentage of CD8 T cells in total TCRαβ⁺ T cells, (B) percentage of CD8 Treg cells in the TCRαβ⁺PLZF⁺CD8αα⁺Vα7.2⁻CD161^{+/-} gate and (C) percentage of CD244⁺CD11c⁺ CD8 Treg in PBMC of healthy individuals and RA patients. (D) CD8 Treg from RA patients significantly secrete more Granzyme B than healthy controls (n=10-11) (**p≤0.01, Mann Whitney test). Scatter plots show Geo MFI values for Granzyme B, IL-17A, Perforin, CD244 and CD11c expression. (E) High frequency of circulating Granzyme B⁺ and CD244⁺ CD8 Treg in RA patients. Scatter plots show percentage of CD8 Treg that were positive for Granzyme B, IL-17A, Perforin and CD11c. PBMC were analyzed by multi-parameter flow cytometry. Each dot represents an individual donor. Results shown as mean±SEM.

FIGS. 19A-D. The frequency of CD8 Treg is increased in PBMCs from systemic lupus erythematosus (SLE) patients. Cumulative data of (A) percentage of CD8 T cells in total TCRαβ⁺ T cells and (B) percentage of CD8 Treg cells in the TCRαβ⁺PLZF⁺CD8aa⁺ Vα7.2⁻CD161^{+/-} gate. (C) Circulating CD8 Treg from SLE patients (n=4) significantly secrete more IFNγ, Granzyme B and TNFα than healthy controls. Scatter plots show Geo MFI values in CD8 Treg for IFNγ, Granzyme B, TNFα, IL-17A and IL-4. (D) CD8 Treg from SLE patients have significantly high expression of RORγt. No differences were found between groups regarding to IL-18Rα, CCR6 and CXCR6 expression. Scatter plots show Geo MFI values in CD8 Treg for IL-18Rα, CCR6, CXCR6 and RORγt. PBMC were analyzed by multi-parameter flow cytometry Each dot represents an individual donor. Results shown as mean±SEM. (E) High frequency of circulating TNFα⁺ CD8 Treg in SLE patients. Scatter plots depict percentage of CD8 Treg are positive for the corresponding cytokine. PBMC were analyzed by multi-parameter flow cytometry. All data are represented as mean±SEM. *p<0.05, Mann-Whitney test.

FIGS. 20A-C. No significant alterations in the frequency of CD8 Treg in PBMCs from head and neck squamous cell carcinoma patients. Cumulative data of (A) percentage of CD8 T cells in total TCRαβ⁺ T cells, (B) percentage of CD8 Treg cells in TCRαβ⁺PLZF⁺CD8ααVα7.2⁻CD161^{+/-} gate and (C) percentage of CD244⁺CD11c⁺ CD8 Treg from PBMC of healthy individuals and head and neck squamous cell carcinoma (HNSCC) patients (n=5). Each dot represents an individual donor. Results shown as mean=SEM.

FIGS. 21A-D. A significant increase in the frequency of circulating CD8 Treg in PBMCs from chronic melanoma patients. Cumulative data of (A) percentage of CD8 T cells in total TCRαβ⁺ T cells, (B) percentage of CD8 Treg cells in TCRαβ⁺PLZF⁺CD8ααVα7.2⁻CD161^{+/-} gate and (C) percentage of CD244⁺CD11c⁺CD8 Treg from PBMC of healthy individuals and melanoma patients. The frequency of CD8 Treg in PBMC from melanoma patients (n=13) was significantly increased compared to healthy controls. PBMC were analyzed by multi-parameter flow cytometry. Each dot represents an individual donor. Results shown as mean±SEM. (D) Identification of infiltrating CD8 Treg into human melanoma tumor by flow cytometry.

FIG. 22. A working model of the negative feedback regulatory mechanism mediated by a novel innate-like PLZF⁺CD8αα⁺TCRαβ⁺ Treg population (CD8 Treg) enriched in liver and colon of naïve mice. (a) During chronic inflammation, CD8 Treg recognize Qa-1-expressing activated CD4⁺ T cells; (b) the CD8 Treg TCR bind to the non-classical Qa-1-bound peptides on the surface of activated Car T cells; (c) following recognition, CD8 Treg mediate release of granzyme B and perforin and induce apoptosis in the target activated CT4⁺ T cells. Since naïve CD4 T cells do not express Qa-1 molecules on the surface, they are not susceptible to CD8 Treg-mediated apoptosis allowing the immune response to proceed.

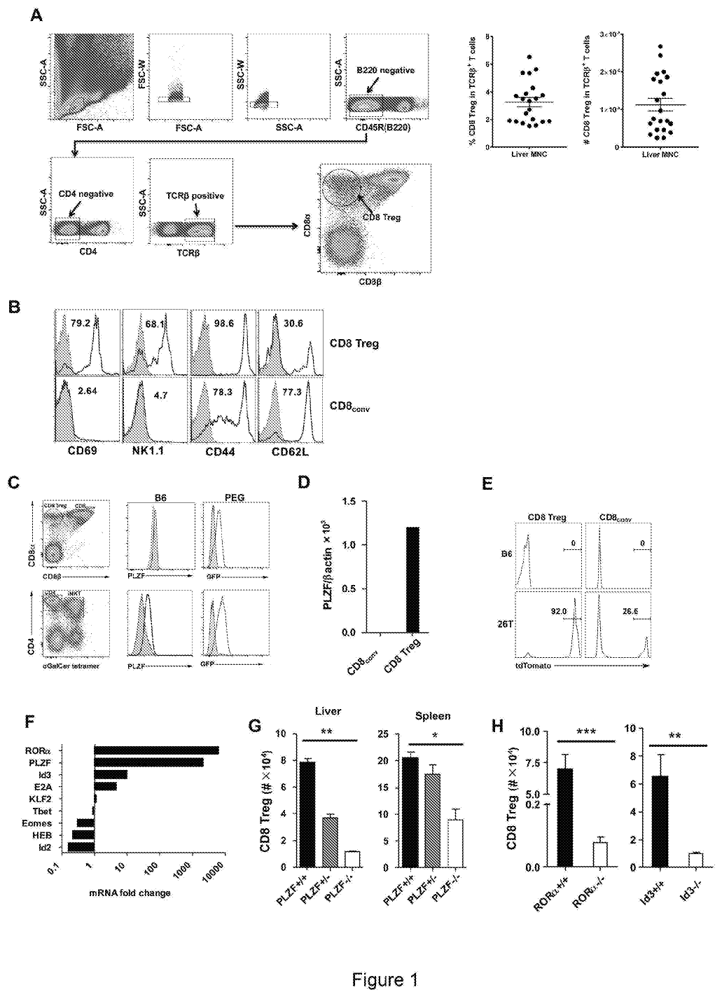

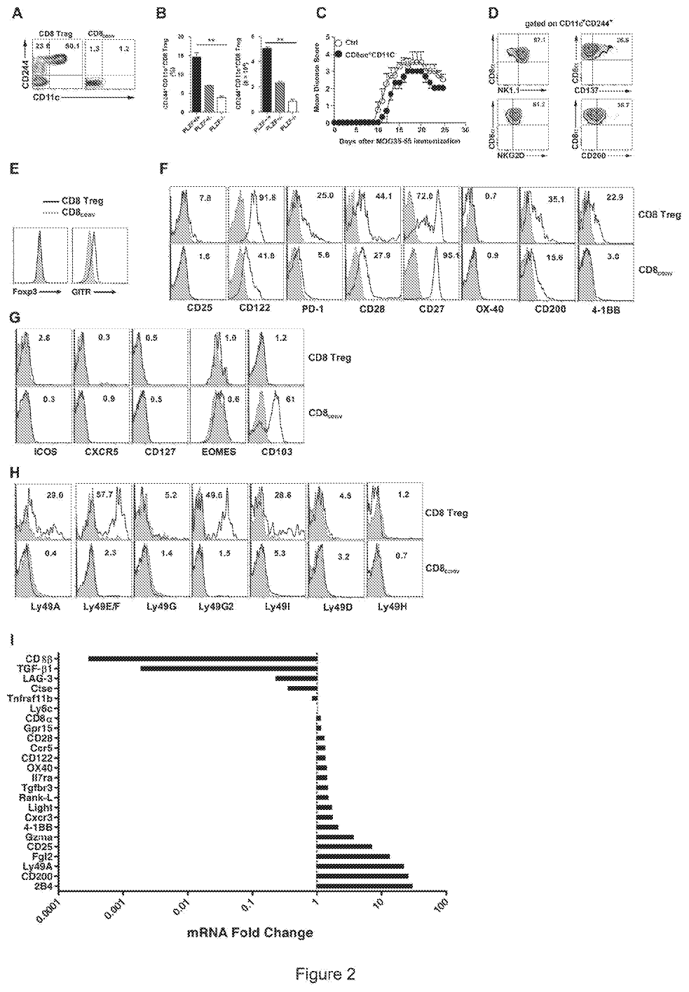

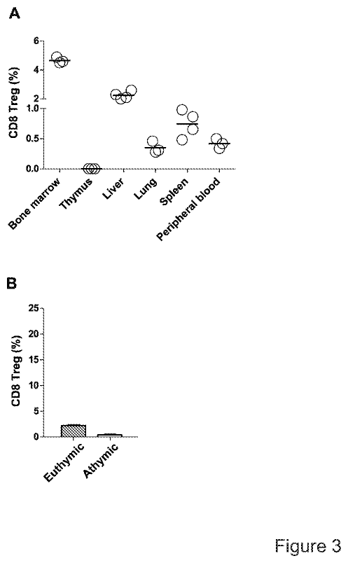

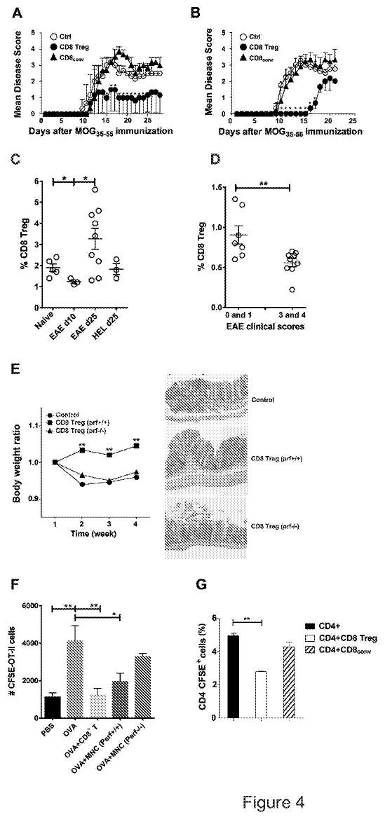

### DETAILED DESCRIPTION

### Definitions

One of ordinary skill in the art will appreciate that an antibody consists of four polypeptides: two identical copies of a heavy (H) chain polypeptide and two copies of a light (L) chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C) region. The variable regions of each pair of light and heavy chains form the antigen binding site of an antibody.

An antibody, or antigen-binding fragment thereof, can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5:511 (1976); Harlow and Lane (eds), Antibodies: A Laboratory Manual, CSH Press (1988); and C. A. Janeway et al. (eds.), Immunobiology, 5th Ed., Garland Publishing, New York, N.Y. (2001)). In certain embodiments, a human antibody or a chimeric antibody can be generated using a transgenic animal a mouse) wherein one or more endogenous immunoglobulin genes are replaced with one or more human immunoglobulin genes.

The term “antigen-binding fragment” refers to one or more fragments of an antibody that retain the ability to specifically bind to an antigen. Examples of antigen-binding fragments include but are not limited to (i) a Fab fragment, which is a monovalent fragment consisting of the V_{L}, V_{H}, C_{L}, and C_{H1} domains; (ii) a F(ab′)2 fragment, which is a bivalent fragment comprising two Fab fragments linked by a disulfide bridge at the hinge region; and (iii) a Fv fragment consisting of the V_{L} and V_{H} domains of a single arm of an antibody.

### Routes and Formulations

Administration of compositions can be via any of suitable route of administration, particularly parenterally, for example, intravenously, intraarterially, intraperitoneally, intrathecally, intraventricularly, intraurethrally, intrasternally, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, critric, and/or phosphoric acids and their sodium salts, and preservatives.

The compositions alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the compositions alone or in combination with another active agent may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the composition optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The composition optionally in combination with another active compound may also be administered intravenously or intraperitoneally by infusion or injection. Solutions of active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can he brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the antigen(s) and adjuvant(s) optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the invention provides various dosage formulations optionally in combination with another active compound for inhalation delivery. For example, formulations may be designed for aerosol use in devices such as metered-dose inhalers, dry powder inhalers and nebulizers.

Useful dosages can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 uM, which can be effective to kill cancer cells. In managing the patient, the therapy should be initiated at a lower dose and increased depending on the patient's global response.

### Exemplary Embodiments

In one embodiment, a method to identify or detect immune cells having TCαβ+, CD8αα+, Nk1.1+, PLZF+, CD161+, and optionally having one or more of CD11c+, CD137+ CD244+, or one or more of NK-inhibitory receptors is provided. The method includes contacting a sample having mammalian immune cells with a ligand that binds CD8αα, a ligand that binds NK1.1, a ligand that binds PLZF, and a ligand that binds CD161, and optionally a ligand that binds CD11c, a ligand that binds CD137, a ligand that binds CD244, a ligand that binds TCRαβ, or a ligand that binds NK-inhibitory receptors; and identifying or detecting an amount of a population of cells CD8αα+, Nk1.1+, PLZF+, and CD161+, and optionally having one or more of CD11c+, CD137+ CD244+ TCRαβ+, or one or more of NK-inhibitory receptors. In one embodiment, the cells are identified using antibodies specific for TCRαβ,CD8αα, Nk1.1, PLZF, or CD161, and optionally antibodies specific for one or more of CD11c, CD137, CD244, or one or more of NK-inhibitory receptors. In one embodiment, the cells are human cells. In one embodiment, the method further comprises isolating the identified cells. In one embodiment, the method further comprises expanding the isolated cells. In one embodiment, the cells are from a patient with an autoimmune disease. In one embodiment, the isolated cells are cultured with IL-2, IL-15, Qa-1/HLA-E binding peptides, anti-CD 3 antibodies or anti-CD137 antibodies, or any combination thereof.

In one embodiment, a method to decrease the number of immune cells having TCRαβ, CD8αα, Nk1.1, PLZF, and CD161, and optionally having one or more of CD11c, CD137, CD244, or one or more of NK-inhibitory receptors in a mammal, is provided. The method includes administering to the mammal an effective amount of one or more antibodies specific for CD8αα, specific for Nk1.1, specific for PLZF, or specific for CD161, or a combination thereof, and optionally a composition comprising one or more antibodies specific for CD11c, specific for CD137, specific for CD244, specific for TCRαβ, or one or more of NK-inhibitory receptors, or a combination thereof.

Also provided is a method to prevent, inhibit or treat cancer in a mammal, comprising: administering to the mammal an effective amount of one or more antibodies specific for CD8αα, specific for Nk1.1, specific for PLZF, or specific for CD161, or a combination thereof, and optionally a composition comprising one or more antibodies specific for CD11c, specific for CD137, specific for CD244, specific for TCRαβ, or specific for one or more of NK-inhibitory receptors, or a combination thereof. In one embodiment, the cancer is neck or head cancer. In one embodiment, the cancer is melanoma.

Further provided is a method to prevent, inhibit or treat autoimmune disease in a mammal, comprising: administering to the mammal one or more Qa-1/HLA-E binding peptides, anti-CD11c antibodies, anti-CD3 antibodies or anti-CD137 antibodies, or any combination thereof, in an amount effective to stimulate CD8a+T regs. In one embodiment, the one or more antibodithat are specific for CD11c and CD137, CD11c and CD3, or CD3 and CD137. In one embodiment, the disease is IBD, colitis, lupus or RA. In one embodiment, the disease is an autoimmune liver disease. In one embodiment, the disease is autoimmune hepatitis or primary biliary cirrhosis. In one embodiment, the T or B cells in the mammal with the disease are increased relative to a mammal without the disease.

In one embodiment, a method to prevent, inhibit or treat organ or graft rejection in a mammal is provided. In one embodiment, comprising: administering to the method includes administering to a mammal in need theref one or more Qa-1/HLA-E binding peptides, anti-CD11c antibodies, anti-CD3 antibodies or anti-CD137 antibodies, or any combination thereof, in an amount effective to stimulate CD8α+ T regs.

In one embodiment, the mammal is a human. In one embodiment, the peptide(s), antibodies, or composition is systemically administered.

The invention will be described by the following non-limiting example.

### EXAMPLE 1

The liver has a central role not only in the metabolism and clearance of the diet and intestinal microbe-derived toxins but also mount immunity against infectious agents and cancer. Chronic infections in the liver indicate that several tolerance mechanisms are evolved to protect the hepatic tissue from excessive immune stimulation (1-3). Liver has several residential T cells, B cells and macrophages or Kupffer cells and in steady state immune cells are predominantly in the sinusoidal blood vessels and do not infiltrate into parenchyma. Among T lymphocytes, liver uniquely harbors both conventional T cells and several unconventional T cells (4). The unconventional T cells are comprised of NKT cells, MAIT cells, and gd T cells in the liver. While adaptive T cells are class Ia MHC-restricted, innate-like unconventional T cells are reactive to non-classical MHC-Ib molecules and recognize a class of antigens. Thus, NKT cells and MAIT cells recognize both self and microbial antigens in the context of CD1d and in the context of the MR-1 molecules, respectively and could provide immunity against microbes (5-7). A detailed understanding of cellular and molecular interactions in liver among different innate and conventional T cells involved in the maintenance of immune tolerance is lacking.

T cells are controlled by both intrinsic (e.g., PD1, anergy and exhaustion) and extrinsic cell-based (regulatory T cells or Treg) mechanisms that prevent them from causing excessive tissue damage. An important role for FoxP3+CD4+ Treg has been well-defined, a phenotypic characterization of CD8+ cells with regulatory activity is poorly studied. Although immune regulatory nature of CD8⁺ T cells in homeostasis of cellular and humoral immune responses has been suggested (8-10) (11-13), ability to distinguish them (CD8 Treg) from non-regulatory conventional CD8⁺ T cells (CD8conv) has not sufficiently advanced as happened for CD4 Treg following the discovery of Foxp3 (14). Similar to CD4+Treg, an important role for IL-2 signaling for CD8+ Treg has been shown in mice deficient in IL-2 and IL-2Rb chain (15-17) (18). Regulatory CD8⁺ T cells also have been implicated in various conditions in humans, e.g. transplant survival (19), inflammatory bowel disease (20) and in multiple sclerosis (21-25). In a clinical trial with anti-CD3 mAb (Teplizumab), increased frequency of memory CD8⁺ T cells with regulatory gene expression was found to be associated with a positive clinical response in type 1 diabetes patients (26). Interestingly, IL-2/IL-15Rβ-deficiency in humans also leads to a severe combined immunodeficiency syndrome as observed for IL-2Rα (27).

In this study, for the first time, we have identified that the expression of the promyelocytic leukemia zinc finger (PLZF) transcription factor in a novel population of PLZF⁺TCRαβ⁺CD8αα⁺ Treg (hereafter referred as CD8⁺ Treg) enriched in liver of naive mice and in human peripheral blood distinguishes them from conventional CD8⁺ T cells. CD8⁺ Treg have a unique cell surface phenotype in that they are CD11c+2B4+NKG2D+ and, despite having innate-like features, they are distinct from other innate-like T cells, including NKT cells or mucosal associated invariant T (MAIT) cells. CD8⁺ Treg do not express Foxp3 but they express glucocorticoid-induced tumor necrosis factor-related receptor (GITR), a marker of active Tregs cells (28). A large number of them are dependent upon Qa-1b molecules as a significant reduced frequency is found in Qa-1^{b}−/− mice. They are TAP-independent but dependent upon IL15 signaling for their development. CD8⁺ Treg do not secrete IL-10 or TGFβ but secrete typical cytokines produced by cytotoxic T cells (TNFα, IL-17A) as well as PLZF-driven secretion of both IL-4 and IFNγ similar to that in NKT cells. It is noteworthy that these cells very rapidly secrete large amounts of IL-2 as well. CD8+ Treg shares several features with other unconventional T cells, including expression of memory/activation markers, a common transcription program imprinted by the expression of PLZF and their enrichment in non-lymphoid organs.

### Results

Liver Enriched CD8 Treg are PLZF⁺ and Dependent upon PLZF and Related Transcription Factors

We have identified a subset of TCRαβ⁺CD8⁺ T cells with immune regulatory properties enriched in liver of naïve C57BL/6 (B6) mice that express the homodimer CD8αα and hereafter referred to as CD8 Treg and different from the conventional TCRαβ⁺CD8⁺ T cells (CD8conv) that do not express PLZF and are CD8ab+. CD8 Treg represent ˜3.25% of TCRαβ⁺ T cells (within the B220-CD4-CD8b− gate) in liver mononuclear cells (MNCs) in naïve B6 mice. They are also present in other lymphoid compartments, including bone marrow, spleen, peripheral blood and lung. Although, CD8 Treg are not readily detectable in adult thymus, they are present in the neonatal period (˜0.5%) until day 5 after birth (data not shown). Athymic nude mice are deficient in CD8 Treg, indicating their thymic origin. In contrast to hepatic CD8_{conv}, the majority of CD8 Treg are CD69+ and NK1.1⁺, suggesting an activated profile similar to other innate-like T cells, including NKT cells. Additionally, CD8 Treg are mostly CD44^{high} and CD62L^{low} compared to CD8_{conv}, suggesting a memory phenotype.

Since the promyelocytic leukemia zinc finger (PLZF) transcription factor controls the development of several innate-like T cells, including NKT cells, γδ T cells, MAIT cells, and memory-like CD8⁺ T cells (Savage A K, Immunity 2008; Weinreich M A, Nat Immunol 2010; Gordon S M, JI 2011; Eidson M, PLoS ONE 2011; Fergusson J R, Cell Reports 2014; Marrero I, Front Immunol 2015) we investigated whether CD8 Treg also express PLZF and whether it is involved in their development. We used anti-PLZF mAb for intracellular staining and GFP expression in liver MNCs from B6 and PLZF-eGFP reporter (PEG) mice (Zhang S, Sci Rep 2015), respectively, and found that CD8 Treg are PLZF⁺ but express lower levels PLZF in comparison to iNKT cells. In contrast, CD8_{conv} or CD4 T cells did not express PLZF. Accordingly, PLZF mRNA expression was found exclusively in sorted CD8 Treg but not in CD8_{conv}.

Next, we investigated whether low level of PLZF expression in hepatic CD8 Treg is similar to that recently described adipose tissue-resident iNKT cells (Lynch L, Nat Immunol 2015). We used the fate-mapping experiment with PLZF-Cre x R26T mice, in which cells that express PLZF are permanently labeled for tdTomato (Zhang S, Sci Rep 2015) and found that most CD8 Treg, in the liver of PCre x R26T mice were positive for tdTomato while CD8_{conv} showed only background level of expression.

Next, we examined whether other related transcription factors, which are associated with innate and/or activated/memory T cells, are involved in the development of CD8 Treg. A semi-quantitative analysis of sorted CD8 Treg vs. CD8Conv showed relative upregulation of the retinoic acid-receptor-related orphan receptor alpha (RORα) (Dzhagalov I, J I 2004; Leppkes M, Gastroenterology 2009), DNA-binding protein inhibitor ID-3 (Id3) (Ji Y, Nat Immunol 2011) and immunoglobulin enhancer-binding ⁻factors E12/E47 (E2A) (Mjösberg J, Eur J Immunol 2012) in CD8 Treg whereas the DNA-binding protein inhibitor ID-2 (M2), the T-box transcription factor Eomes and HeLa E-box binding protein (HEB) were down-regulated in comparison to CD8_{conv}. However, T-bet and Kruppel-like factor 2 (KLF2) were not significantly different between CD8 Treg and CD8_{conv}. A critical role of PLZF, RORα and Id3 was confirmed in mice genetically deficient in these transcription factors. PLZF−/−, RORα−/− and Id3−/− mice showed a significant reduction in the number of CD8 Treg. These results indicate that CD8 Treg are PLZF⁺ and are dependent upon PLZF and related transcription factors.

Altogether, we have identified a novel subset of hepatic PLZF⁺CD8αα⁺TCRαβ⁺ Treg that express innate-like activated-memory markers in naïve mice.

Hepatic CD8 Treg have Immune Regulatory Properties and are Physiologically Expanded During an Experimental Autoimmunity

Next, we determined whether CD8 Treg have regulatory potential to control autoimmunity. An equal number of sorted hepatic CD8 Treg or CD8_{conv} from naïve B6 mice were transferred into B6 recipients that were immunized next day with myelin-oligodendrocyte glycoprotein (MOG)₃₅₋₅₅ peptide in completed Freund's adjuvant (CFA) plus pertussis toxin (PTx) (MOG₃₅₋₅₅/CFA/PTx) for the induction of experimental autoimmune encephalomyelitis (EAE). Mice that received CD8 Treg showed a significant suppression of EAE in comparison to those receiving CD8_{conv} or PBS control. To exclude the role of both PLZF⁺CD1d-restricted NKT cells as well as innate-like memory CD8⁺ T cells that depend on IL-4 secreted by NKT cells, hepatic CD8 Treg or CD8_{conv} were sorted from CD1d−/− mice deficient in both populations and used in adoptive transfer experiment as above. Mice that received CD8 Treg but not CD8_{conv} from CD1d−/− mice showed significant protection from EAE. These data indicate regulatory property of CD8 Treg independent of NKT cells or innate-like memory CD8 T cells.

Next, we investigated whether CD8 Treg are physiologically altered in liver during the course of EAE. Thus, B6 mice were immunized with either MOG₃₅₋₅₅ for the EAE induction or an irrelevant peptide derived from hen egg lysozyme (HEL) and hepatic CD8 Treg were analyzed on day 10 at the onset of disease (EAE d10) or at day 25 during the recovery phase of the disease (EAE d25). CD8 Treg were significantly expanded during the recovery phase (day 25) but not at the onset of EAE. In contrast, CD8 Treg numbers were not altered in non-diseased mice immunized with HEL peptide. Next, we determined whether the CD8 Treg expansion inversely correlates with the severity of EAE. Interestingly, a significantly higher expansion of CD8 Treg occurred in mice with low clinical scores (0-1) compared with those with severe clinical scores (3-4). These data indicate CD8 Treg are physiologically expanded during recovery phase of EAE and their higher numbers correlating with less severe disease suggest their physiological role in spontaneous recovery as suggested earlier for the CD8 T cells.

To further investigate whether the immune regulation mediated by CD8 Treg requires other lymphocytes and whether a cytotoxic mechanism is involved in this regulation, we used the CD4⁺CD25⁻CD45RB^{high} T cells mediated colitis into Rag1−/− mice and co-transfered CD8 Treg from B6 or perforin−/− mice, ARag1−/− recipients that received CD8 Treg from perforin−/− mice developed colitis similar to the control mice whereas mice that received CD8 Treg from perforin+/+ mice were significantly protected as indicated by increase in body weight ratio and histological analysis of colon using H&E staining. These results indicate that CD8 Treg use a perforin-dependent mechanism to control autoimmunity mediated by activated CD4⁺ T cells.

To further examine the mechanism of regulation, we used both in vivo and in vitro assays to determine the fate of CFSE-labeled OT-II CD4⁺ T cells specific for a chicken ovalbumin (OVA)₃₂₃₋₃₃₉ peptide. Thus, CFSE-labeled OT-II CD4⁺ cells were transferred to naïve B6 mice alone or co-transferred with positive-selected CD8⁺ T cells or liver MNCs from B6 or perforin−/− mice. Next day, recipients were i.p. challenged with OVA₃₂₃₋₃₃₉ peptide or PBS and, 4 days later, proliferation of CFSE-labeled OT-II CD4⁺ T cells was analyzed by flow cytometry. Enriched CD8⁺ T cells and liver MNCs from B6 (perforin+/+) but not from perforin−/− mice significantly suppressed OVA proliferation of CD4⁺ T cells as indicated by reduced recovery of CFSE-labeled OT-II CD4⁺ T cells. Next, we examined whether CD8 Treg are able to inhibit proliferation of activated CD4⁺ T cells in vitro. Sorted CFSE-labeled CD4⁺ T cells alone or co-cultured with sorted hepatic CD8 Treg or CD8_{conv} were in vitro stimulated with plate-bound anti-CD3 mAb. CD8 Treg but not CD8_{conv} were able to inhibit significantly the expansion of CD4⁺ T cells. Collectively, these results indicate that CD8 Treg have regulatory activity and able to control activation/expansion of disease-causing CD4 T cells. Moreover, the addition to the culture of anti-Qa-1^{b} mAb but not anti-MHC class I mAb significantly blocked the suppressive function of CD8 Treg (data not shown), suggesting a dependence on Qa-1^{b} molecules.

### Hepatic CD8 Treg Secrete Typical PLZF-Driven Cytokines and are Dependent on IL-15/IL-2Rβ Signaling for Their Development

To further understand the mechanism of regulation by CD8 Treg, we determined the cytokine secretion profile of sorted hepatic CD8 Treg and compared to the sorted CD8_{conv} from naïve B6 following in vitro stimulation with the plate-bound anti-CD3 mAb for 96 hr and cytokine secretion measured in culture supernatants. As shown in FIG. 42A, CD8 Treg secreted typical pro-inflammatory cytokines produced by cytotoxic T cells (TNFα, IL-17A and IL-6) as well as PLZF-driven secretion of both IFNγ and IL-4. Notably, CD8 Treg secreted significantly higher levels of IL-2 within 24 hr while CD8_{conv} secreted only a minimal amount. Importantly, CD8 Treg do not secrete the suppressive cytokine IL-10 and the expression of TGFβ was significantly down-regulated in these cells. Consistently, CD8_{conv} showed a typical cytotoxic T cell profile, including secretion of IFNγ and TNFα.

We next determined the cytokine signaling requirement for the survival and/or development of CD8 Treg. Hepatic CD8 Treg were significantly reduced in both CD122−/− and IL-15−/− mice in comparison to B6 mice. Considering that CD122, the β chain receptor for IL-2 and IL-15, is essential for CD8⁺ T cell response to IL-15, these results indicate that IL-15 signaling is necessary for CD8 Treg development. In contrast, CD8 Treg are not affected in CD25−/−, IL-7−/−, IL-6−/−, and IFNγ−/− mice. Surprisingly, CD8 Treg are T-bet independent and are not reduced in T-bet−/− mice.

We next determined the requirement of other immune molecules for CD8 Treg development. As expected, hepatic CD8 Treg were absent in CD8α−/− mice but their frequency was significantly increased in CD8β−/− mice, confirming the expression of the CD8α chain in CD8 Treg. No significant changes in the frequency of hepatic CD8 Treg was observed in CD1d−/−, Jα18−/− and μMT−/− mice, indicating that NKT cells and B cells are not required for CD8 Treg development. Importantly, a significantly reduced frequency of CD8 Treg in Qa-1^{b}−/− mice indicate that a proportion of CD8 Treg are restricted by Qa-1^{b} molecules. Since Qa-1-restricted T cells can be either TAP-dependent or TAP-independent, no differences in the frequency CD8 Treg between TAP1−/− and B6 mice indicate that TAP-dependent antigen processing is not required for the development of CD8 Treg.

The regulatory function hepatic CD8 Treg and their dependence on Qa-1 expression was further confirmed in a model of acute immune-mediated hepatitis using concanavalin A (ConA) (Maricic I et al, J I 2014; Heymann F et al, Lab Anim 2015). ConA-induced hepatitis was significantly exacerbated in Qa-1−/− mice in comparison to B6 mice, as indicated by liver damage using ALT levels and H&E staining.

### Hepatic CD8 Treg are Polyclonal and Use Diverse TCR α and β Chains

Since PLZF-dependent innate-like T cells, including NKT and MAIT cells, often used limited TCR, we examined the TCR repertoire of CD8 Treg using available anti-TCR-Vα or Vβ antibodies, RT-PCR and high-throughput sequencing. Flow cytometry-based TCR Vβ repertoire analysis of CD8 Treg indicate a diverse Vβ chain usage with a bias toward the use of Vβ8.1/8.2, Vβ5.1/5.2, Vβ8.3 and Vβ11 chains. We further investigate the TCR repertoire of CD8 Treg by high-throughput sequencing of the TCR β chain CDR3 region of sorted CD8 Treg as described (Marrero I, PLoS ONE 2013; Marrero I, Mol Immunol 2016). This confirmed that CD8 Treg expressed a diverse TCRβ repertoire with great variations of TCR sequences and preferential usage of TRBV13-3/13-2 (Vβ8.1/8.2), TRBV20 (Vβ15), TRBV12-1/12-2 (Vβ5.1/5.2), TRBV5 (Vβ1) and TRBV4 (Vβ10). Additionally, TRBJ usage, another indicator of the heterogeneity, was also diverse and almost similar among CD8 Treg and CD8_{conv}, except for an increase in Jβ2.5 usage by CD8 Treg. CD8 Treg also showed a skewed bell-shaped distribution of CDR3 lengths with a peak at 39 nucleotides while CD8_{conv} show a more typical Gaussian-like distribution with a peak at 42 nucleotides. Furthermore, TCR Vα analysis of CD8 Treg by FACS and RT-PCR revealed diverse Vα usage similar to that in CD8_{conv}. Together, these data indicate that the TCR repertoire of the hepatic CD8 Treg in naïve mice is polyclonal.

### Hepatic CD8 Treg are CD11c+ and CD244+ and Express Several Markers Typical of Innate-Like T Cells

To further differentiate CD8 Treg from CD8_{conv}, we determined the expression of different cell surface markers using flowcytometry and quantitative RT-PCR. A large proportion of CD8 Treg but not CD8_{conv} CD11c. In addition, the majority of CD8 Treg express CD244 (2B4). Co-staining of CD8 Treg indicates that all CD11c⁺ CD8 Treg are also CD244⁺ while none of the CD8_{conv} express CD11c or CD244. Next, we determined whether CD8 Treg co-expressing CD244 and CD11c (CD244+CD11c+) are dependent on PLZF. Both percentage and numbers of CD244⁺CD11c⁺ CD8 Treg were significantly reduced in PLZF−/− mice. Consistent with the correlation of regulatory activity with CD11c expression in Treg, CD11c-bead depletion resulted in loss of CD8 Treg ability to control EAE upon adoptive transfer (data not shown). Thus, CD8 Treg have distinguishing features, they are NK1.1+PLZF+, CD244⁺ and CD11c⁺.

Next we determined the expression of FoxP3 in CD8 Treg. Similar to CD8_{conv}, CD8 Treg were negative for FoxP3 but do express glucocorticoid-induced tumor necrosis factor-related receptor (GITR), also present on CD4 Treg (Ronchetti S, J Immunol Res 2015). Consistent with the role of IL-15 signaling, CD8 Treg were CD122(IL-2Rβ)^{high} CD25(IL-2Rα)^{low}, similar to innate-like CD8⁺ T cells (Walzer T, J I 2002), and most of them were CD27^{high}, marker associated with memory CD8⁺ T cells. Interestingly, CD8 Treg were CD28⁺, PD-1⁺ and also express another regulatory signaling molecule CD200. CD8 Treg were negative for OX-40. Notably, CD8 Treg also expressed low levels of 4-1BB (CD137), a co-stimulatory molecule belonging to the TNF-receptor superfamily 9 and expressed only on activated T cells. In addition, CD8 Treg did not express ICOS, CXCR5, Eomes as well as CD103, the α-chain of the αEβ7 integrin that characterize tissue-resident memory (TRM) T cells (Ariotti S, Adv Immunol 2012). CD8 Treg are not typical memory cells as they do not express CD127 (IL-7Rα), which is required for memory CD8 T cells.

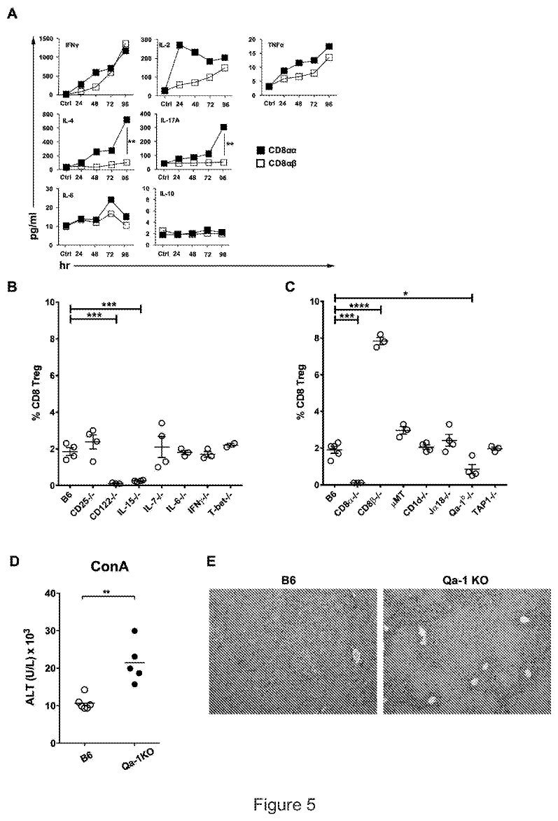

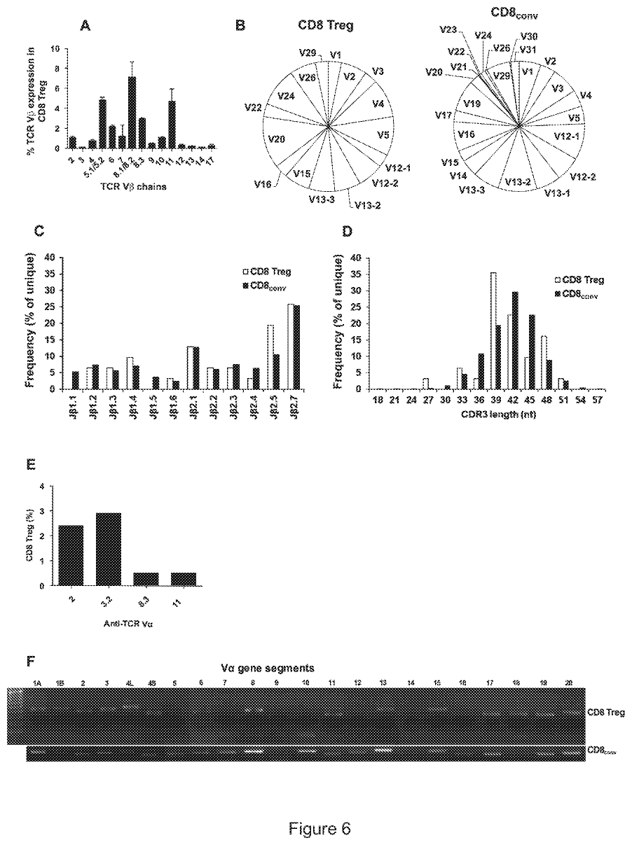

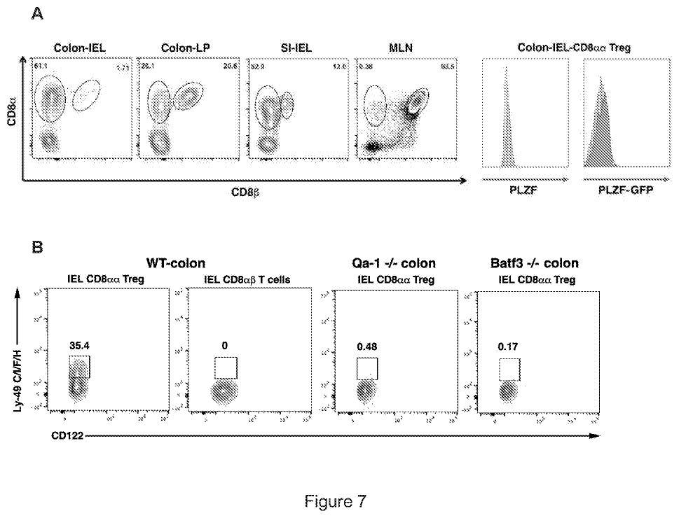

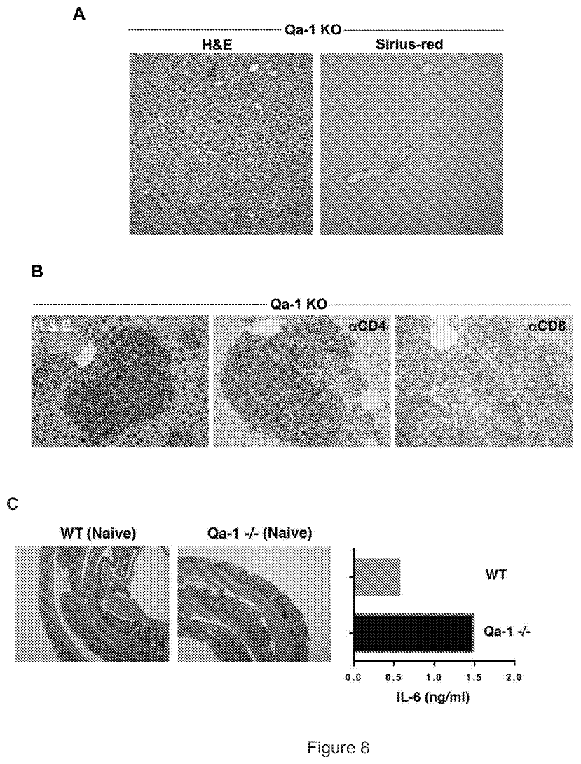

Since CD8 Treg are PLZF⁺, we determined the expression of other markers associated with innate cells. As shown in FIG. 5F, CD8 Treg express only NK inhibitory receptors (Ly49E/F>Ly49G2>Ly49A>Ly49I>Ly49G), but not activating receptors (Ly49D and Ly49H). Consistent with an innate-like behavior, CD8 Treg also express NKG2D.

To further confirm their phenotype, we analyzed the expression of a set of selected genes by semiquantitative RT-PCR on sorted hepatic CD8 Treg vs. CD8_{conv}. Consistent with the flow cytometry analysis, CD8 Treg showed significant down-regulation of CD8β and up-regulation of CD8α, CD244 (2B4), CD200, Ly49A, Granzyme A (Gzma), 4-1BB, CD25, CD122 and CD28. Interestingly, Fibrinogen-like 2 (Fgl2), which is a negative regulator of the immune response was up-regulated in CD8 Treg. Interestingly, other genes associated with CD4 Treg, including TGF-β1 and Lymphocyte-activation gene 3 (LAG-3), were down-regulated in CD8 Treg in comparison to CD8_{conv}.

PLZF⁺ CD8 Treg are also Present in Human Peripheral Blood and Have Several Features Similar to the Murine Counterpart

To determine whether PLZF+ CD8 Treg are also present in humans, we examined peripheral blood mononuclear cells (PBMC) from 23 healthy individuals by multiparameter flow cytometry using a gating strategy based on PLZF expression and exclusion of circulating mucosal-associated invariant T (MAIT) cells, which have been defined by usage of the semi-invariant canonical TCRα chain Vα7.2/Jα33 and high expression of CD161 (CD161⁺⁺) (Porcelli S, JEM 1993). Following this strategy, in human PBMC, CD8 Treg were defined as PLZF⁺TCRαβ⁺CD8α⁺CD8β⁻T cells that express low to intermediate levels of CD161 (CD161^{+/-}). Human circulating CD8 Treg represent ˜0.24% (±SEM 0.06) of the total TCRαβ⁺ T cells and ˜0.87% (±0.21) of the total CD8⁺ T cells.

Next we used flowcytometry to analyze human CD8 Treg directly ex vivo without stimulation using combination of mAbs specific for cell surface and intracellular molecules as well as cytokines. Similar to murine CD8 Treg, CD244 (2B4) and CD11c expression was significantly higher in circulating CD8 Treg than in both CD8_{conv}. Notably, circulating CD8 Treg also secrete higher levels of Granzyme B than both CD8_{conv} and MAIT cells. Similar to murine CD8 Treg, human CD8 Treg also secrete IFNγ, TNFα, IL-4 and IL-17A. Furthermore, the expression of IL-18 receptor α subunit (IL-18Rα1), RORγt, CXCR6 and CCR6, known to be up-regulated in MAIT cells were significantly reduced in CD8 Treg. Similar to murine CD8 Treg, a subset of human CD8 Treg also co-express CD244 and CD11c (CD244⁺CD11c⁺) that was significantly higher in CD8 Treg than in MAIT cells (15.7%±3.0 vs 1.4%±0.7). Interestingly, ˜69% of CD244⁺CD11c⁺ CD8 Treg secrete both Granzyme B and perforin.

Next we determined the TCRβ repertoire of circulating CD8 Treg by high-throughput sequencing and compared it to the repertoire of CD8_{conv}. We obtained 794 productive unique sequences from sorted CD8 Treg and 12,244 productive unique sequences from sorted CD8_{conv}. Similar to murine CD8 Treg, circulating CD8 Treg exhibited a non-restricted usage of multiple TRBV gene segments that resemble the TCRβ repertoire of CD8_{conv}. Several TRBV gene segments, including TRBV6 (Vβ13), TRBV7( ), TRBV5( ), TRBV27( ) and TRBV20 (Vβ2), were expressed at almost similar frequencies by both subsets. Both CD8 Treg and CD8_{conv} do not show any preferential usage of specific TRBJ gene segment and showed a Gaussian-like CDR3β length distribution profiles. Collectively, these results indicate that circulating human CD8 Treg express a diverse and polyclonal TCRβ repertoire. The proportion of circulating CD8 Treg within total PLZF⁺CD8αα⁺CD161^{+/-} T cells was ˜12.7% (±2.2) that represent ˜2×10³ (±0.6×10³) CD8 Treg. In contrast, MAIT cells represent ˜2.4% (±0.58) of the total TCRαβ⁺ T cells and ˜8.2% (±2.0) of the total CD8⁺ T cells (data not shown)

### EXAMPLE 2

We have identified an innate-like memory PLZF+ CD8alphaalpha⁺TCRalphabeta⁺ Treg enriched in liver and colon of naïve mice, hereafter referred to CD8 Treg, expressing the promyelocytic leukemia zinc finger (PLZF) transcription factor that distinguishes it from conventional CD8⁺ T cells. Our long-term objective is to define the unique phenotype and the regulatory mechanism(s) mediated by CD8 Treg that target activated, but not naive T cells in a negative feedback regulation mechanism. We propose to characterize the phenotype and mechanism of regulation mediated by CD8 Treg in murine models of dextran sodium sulfate (DSS)-induced colitis in B6 mice and CD45Rb^{High}CD4⁺ T cells-induced colitis in Rag1−/− mice.

These investigations are supported by key preliminary data demonstrating that CD8 Treg: 1) are enriched within the colonic CD8αα⁺TCRαβ⁺ IEL population and are memory-like (CD44^{high}CD62L^{low}), activated (CD122⁺CD25⁺CD69⁺), innate-like (NK1.1⁺) cells that express NK-inhibitory (Ly49C/I/F/H) but not NK-activating receptors; 2) are distinct from MAIT or NKT cells; 3) do not secrete IL-10 and TGF-b but express features of cytotoxic CD8 T cells with enhanced perforin/granzyme B expression; 4) protect Rag1−/− mice from CD4⁺ T cells-induced colitis; and 5) are Qa-1b restricted and can be induced with a Qa-1b-binding TCR leader peptide and protect DSS-induced colitis in a Qa-1b-dependent manner. We propose the central hypothesis that PLZF⁺CD8 Treg, displaying a unique phenotype, play a critical role in the control of inflammation in gut in a negative feedback regulatory mechanism. The following specific aims are designed to test this hypothesis in ongoing investigations:

To characterize cell surface markers and gene expression profile of colonic CD8 Treg: Our hypothesis is that colonic CD8 Treg (PLZF⁺TCRalphabeta⁺NK1.1⁺CD44⁺CD122⁺ expressing NK-inhibitory receptors) are PLZF-dependent and have a transcriptional signature similar to CD8 Treg in the liver.

To determine regulatory properties, induction and mechanism of regulation of colitis by colonic CD8 Treg. We will test the hypothesis that CD8 Treg use cytolytic mechanism to control colitis in a Qa-1b-dependent manner and that CD8 Treg can be activated/expanded following immunization with Qa-1b-binding peptides. Also CD8 Treg can inactivate or kill key dendritic cell populations involved in colitis.

The proposed studies will be important for a greater understanding of the phenotype and function of this newly discovered CD8 Treg population in colon and will have important implications for novel experimental therapeutics in IBD.

Colonic CD8 Treg have a similar phenotype and function: Consistent with earlier data, CD8αTCRαβ+ t cells are present in both the small intestine (SI) and colon. It also has been shown that SI CD8ααTCRαβ T cells and not conventional CD8αβTCRαβ T cells are regulatory and maintain gut homeostasis. We propose that colonic CD8 Treg with a phenotype similar to liver (PLZF⁺, NK1.1⁺, expressing NK-inhibitory receptors and memory phenotype, CD44⁺CD122⁺) are present within the CD8ααTCRαβ⁺ intraepithelial lymphocytes (IEL) population.

Interestingly, although total CD8ααTCRαβ⁺ T cells are not affected in Qa-1b−/− mice, the frequency of CD8 Treg (Ly49C/I/F/H+) is significantly reduced similar to that in the liver, indicating their Qa-1b-MHC restriction. Next, we have determined whether CD8 Treg are able to protect from CD4⁺ T cell-induced colitis in Rag1−/− mice. Adoptive transfer of CD8 Treg protect Rag1−/− mice from Ca4⁺CD45Rb^{high} T cell-induced colitis. Thus, if CD8 Treg are important in the maintenance of gut homeostasis, we predict that Qa-1b−/− mice, lacking CD8αα Treg, should become more susceptible or sensitive to dextran sulfate sodium (DSS)-induced colitis.

A Qa-1b-Binding Peptide Induces CD8 Treg and Protect DSS Colitis:

Since CD8 Treg are Qa-1b-restricted, we have synthesized several peptides with the potential Qa-1b-binding motif derived from the conserved regions of the TCR Vβ chains, heat shock proteins (HSP), and QDM. Next, we have determined their potential to induce Qa-1-dependent CD8⁺ T cell response in naïve B6 mice. We found 6 peptides that are able to bind to the Qa-1 molecule (in in vitro Qa-1b folding assays) and induce a CD8⁺ T cell proliferative response (CFSE-dilution) in naïve B6 mice. Among these peptides, we found that one peptide, p8.2L, derived from the leader sequence of the TCR Vβ8.2 chain, is able to induce CD8 Treg and significantly protect mice from DSS-induced colitis as shown by the cumulative colitis scores and histology. In peptide-treated mice, the secretion of pro-inflammatory cytokines, like IL-6, TNF-α□□IFNγ and IL-17A, decreased significantly in comparison to the control mice. Next, we determined whether the peptide-induced protection is dependent upon the presence of Qa-1 molecules. While peptide treatment significantly protected WT B6 mice, Qa1b−/− mice are not protected from colitis. Collectively, our data suggest that CD8 Tregs are involved in a dominant immune regulatory mechanism able to control both DSS-induced colitis, which is primarily mediated by innate cells, and CD45Rb^{high}CD4⁺ T cell-induced colitis, which is mediated by adaptive Th1/Th17 cells. These preliminary findings are quite significant as CD8 Treg are also present in humans. Using a multi parameter flow cytometry analysis, we have also defined a CD8 Treg population in PBMCs from healthy donors that are PLZF⁺TCRαβ⁺CD8αα⁺CD161^{+(int)} with a memory phenotype expressing NK-inhibitory receptors similar to the phenotype in mice.

Our hypothesis is that colonic CD8 Treg (PLZF⁺TCRαβ⁺NK1.1⁺CD44⁺CD122⁺ expressing NK-inhibitory receptors) are PLZF-dependent and have a transcriptional signature similar to CD8 Treg in the liver.

To Determine the Cell Surface Phenotype of Colonic CD8 Treg:

Rationale: We will identify whether colonic CD8 Treg share common cell surface markers with the newly discovered PLZF⁺CD8αα Treg enriched in liver (Preliminary data & manuscript in preparation). Our preliminary data show the presence of CD8 Treg in colon and enrichment in the IEL compartment in B6 mice. As observed for hepatic CD8 Treg, we will investigate whether colonic CD8αα T cells express characteristic innate-like and memory markers, including CD122, CD11c, NK1.1, NK-inhibitory receptors, etc. Most importantly, we will determine whether colonic CD8 Treg are PLZF⁺, which is found to be crucial in the development of hepatic CD8 Treg and their innate-like features.

Experimental Strategy: We have standardized the isolation of IEL and LP fractions from mouse colon using the isolation kit from Miltenyi Biotech. Briefly, following removal of mesenteric lymph nodes and stool, colon will be cut longitudinally and transversely into small pieces and washed twice in HBSS solution without calcium and magnesium containing 10 mM HEPES, 5 mM EDTA, 5% FBS, 1 mM DTT using the MACSmix tube rotator at 37° C. The washed fluid is the FEL fraction. Following another wash with only HBSS without calcium and magnesium containing 10 mM HEPES, colon pieces will be treated with an enzyme mixture following manufacturer's protocol at 37° C. for 30 mM. Finally, the enzyme-treated colonic pieces will be dissociated into single cell suspension using the gentleMacs tissue dissociator. This is the LP fraction. The IEL and the LP fractions will be labeled with different fluorescent labeled mAbs to identify CD8 Treg within the TCRβ⁺CD4⁻B220⁻CD45⁺ gate and their CD8αβ counterpart using flow cytometry. We will include staining for several activation markers, NK cell-specific markers, memory T cell markers as well as CD11c expression and compare these profiles to conventional CD8αβ T cells as well as to CD8 Treg derived from liver.

To determine the Gene Transcript Signature of Colonic CD8 Treg for the Identification of Cell-Surface Markers and Functional Pathways.

Rationale: We hypothesize that a distinct gene expression profile in this novel CD8 Treg will further help to define not only their cell-surface markers but also the molecular pathways for their identification, development and function. Using cluster analysis program, we will classify these genes into functional molecular classes, such as cell surface molecules and molecules involved in transcription signaling, cell death, metabolism, etc. Since memory program also encompasses changes in a broad set of cellular pathways, including signal transduction, survival, apoptosis, cell cycle regulation, metabolism, nuclear function, and cytoskeletal organization, we will compare these gene signatures between gut- and liver-derived CD8αα Treg as well as tissue-resident memory T cells and virtual memory T cells that are also responsive to IL-15.

Experimental Strategy: We will perform RNA sequencing for transcriptome profiling of CD8 Treg and conventional CD8αβ T cells sorted from colon tissues of naïve B6 mice. It is likely that both populations should share many genes associated with CD8 T cells, but they will have distinct global gene expression patterns owing to the innate-like properties of CD8 Treg. We will define shared genes and those differentially expressed either up-regulated or down-regulated in CD8 Treg in comparison to CD8αβ T cells under stringent statistical analysis (p value<0.05). The candidate genes will be validated by either real-time PCR with specific primers designed using web-based programs as before or at the protein level using available antibodies for western blotting and flow cytometry. Sorted CD8 Treg and CD8αβ T cells will be used for the extraction of total RNA using the RNAeasy Micro Kit (Qiagen) followed by its integrity analysis using Agilent RNA 600 Nano Kit (Agilent). Poly-A mRNA enrichment will be used for library preparation and 50 bp-reads will be generated by sequencing on an Illumina HiSeq4000 analyzer using the TruSeq v3 Cluster kit at the UCSD Institute for Genomic Medicine core facility. RNA sequence data will be analyzed by the Bioinformatics core facility at UCSD and the log2 RPKM (Read per kilobase per million) will be quantile normalized. The R statistical software will be used to calculate differentially expressed genes between CD8αα Treg and CD8αβ T cells. Genes satisfying the following criteria will be chosen for analysis: first, the average count is more than 100 in at least one sample group, and second, the global False Discovery Rate (FDR) is controlled at p values of 0.05 with a minimum fold-change of 2. This should generate a CD8αα Treg vs. CD8αβ T cells “signature gene set” for further analysis.

To Determine PLZF Expression and its Role in the Development of Colonic CD8αα Treg.

Rationale: Our preliminary data suggest lower levels of PLZF expression in both hepatic and colonic CD8αα Treg compared to NKT cells. Also, PLZF plays an important role as hepatic CD8αα Treg are significantly reduced in PLZF−/− mice. First, we will determine the expression of PLZF in CD8αα Treg using the fate mapping strategy as well as bone marrow chimeric mice using PLZF−/− mice as described earlier. The absence of CD8αα Treg in nude mice (data not shown) indicate their thymic origin and we hypothesize that following their migration from thymus CD8αα Treg lose PLZF expression similar to the recently described mechanism for adipose tissue-resident NKT cells. Since PLZF⁺ NKT and MAIT cells are also dependent upon IL-15/IL-2Rβ signaling, we will use several gene-deficient mice to determine by flow cytometry whether similar pathways are also crucial for the development of colonic CD8αα Treg. Experimental Strategy: First, we will determine the frequency and absolute numbers of both CD8αα Treg and CD8a0 T cells in colonic IEL and LP of WT B6 and several gene deficient mice, including PLZF−/−, Qa-1−/−, CD122−/− and CD25−/− mice. To determine expression of PLZF using a fate-mapping strategy, we will cross PLZF-Cre mice (which express a bacterial artificial chromosome transgene in which the gene encoding Cre recombinase is knocked into gene encoding PLZF) with Rosa26 f1/f1 mice (which express the fluorescent marker tdTomato and carry a loxP-flanked stop codon at the ubiquitous Rosa26 locus). Therefore, in the resultant PLZF-Cre X Rosa26 f1/f1 mice, cells that express PLZF (and therefore Cre) are permanently tdTomato+. We will examine whether CD8αα Treg from gut, liver and spleen are tdTomato+. We will also use CD8αβ T cells as negative and type I NKT cells as positive controls in these experiments. These mice are available from Dr. Derek Sant'Angelo who will help us with these experiments. A clear pronounced tdTomato staining will confirm that colonic CD8αα Treg also had expressed PLZF during development. Next, since the conventional T cell development is not perturbed in the absence of PLZF as shown in PLZF−/− mice, chimeric mice using CD45.1 and CD45.2 markers will be generated to study CD8αα Treg. First, we will determine whether the requirement for PLZF is intrinsic to CD8αα Treg by transferring PLZF-deficient bone marrow depleted of T cells and B cells into irradiated B6 host and monitoring the presence of CD8αα Treg using FACS. In parallel, we will also transfer bone marrow from WT littermates into B6 host for comparison. If PLZF plays a cell-intrinsic role, PLZF−/− bone marrow transfer may not be able to fully reconstitute B6 host. To further demonstrate the intrinsic role of PLZF, WT B6 mice will be reconstituted with a 50:50 mix of bone marrow cells from PLZF−/− and WT mice and the development of CD8αα Treg analyzed 12 to 16 weeks after transfer by cell surface markers, including CD45.1 vs. CD45.2 to differentiate WT from PLZF−/− cells.

Alternate strategies: As mentioned before, all colonic CD8αα⁻TCRαβ⁺ T cells do not have regulatory properties. Our preliminary data suggest that around 35-50% of CD8αα⁺TCRαβ⁺ T cells express NK-inhibitory receptors (and are CD44⁺CD122⁺ and CD11c⁺, a marker thought to be present on suppressor T cells (29). In addition, the depletion of CD11c⁺ cells from bulk CD8⁺ T cells results in loss of protection from EAE in adoptive transfer experiments (data not shown). Therefore, attempts will be made to sort and compare gene expression between CD11c⁺ Treg and CD11c⁻ populations. If there is a difference it would indicate that within CD8αα T cells only those that are also CD11c⁺ are CD8αα Treg. It is possible that PLZF requirement is not intrinsic to CD8αα Treg. In this case, CD8αα Treg could still develop in chimeric mice generated with bone marrow cells from PLZF−/− mice. However, CD8αα Treg in these chimeric mice may not have fully developed their innate-like properties, including cytokine secretion, NK1.1 expression or expression of other cytotoxicity genes, like perforin and granzyme B. Therefore, we will use flow cytometry to analyze all relevant markers after in vitro stimulation with plate-bound anti-CD3/CD28 mAbs as shown.

To determine regulatory properties, induction and mechanism of regulation of colitis by CD8αα Treg. In this Aim, we design a series of experiments to test the hypothesis that CD8αα Treg use cytolytic mechanism to control colitis in a Qa-1b-dependent manner and that CD8αα Treg can be activated/expanded following immunization with Qa-1b-binding peptides.

To Determine the Role of Perfornin/Granzyme B in the Regulation of Colitis.

Rationale: Our preliminary data indicate a regulatory role of CD8αα Treg in both CD4-induced and DSS-induced colitis. We hypothesize that CD8αα Treg utilize cytolytic mechanism to kill activated target CD4⁺ T cells based upon the following observations: (a) they do not secrete detectable levels of suppressive cytokines, such as IL-10 and IGFβ; but produce cytokines typical of cytotoxic CD8 T cells and NKT cells; (b) RT-PCR analysis show that CD8αα Treg express enhanced levels of perforin and granzyme B, but not granzyme A; (c) earlier data using bulk CD122⁺CD8⁺ T cells or cloned TCR-peptide-reactive CD8⁺ T cells show killing of activated target cells. Since there is similarity between hepatic CD8αα Treg and colonic CD8αα Treg in all the features analyzed so far, we propose to compare whether both Treg populations use similar regulatory mechanisms involving perforin/granzyme B.

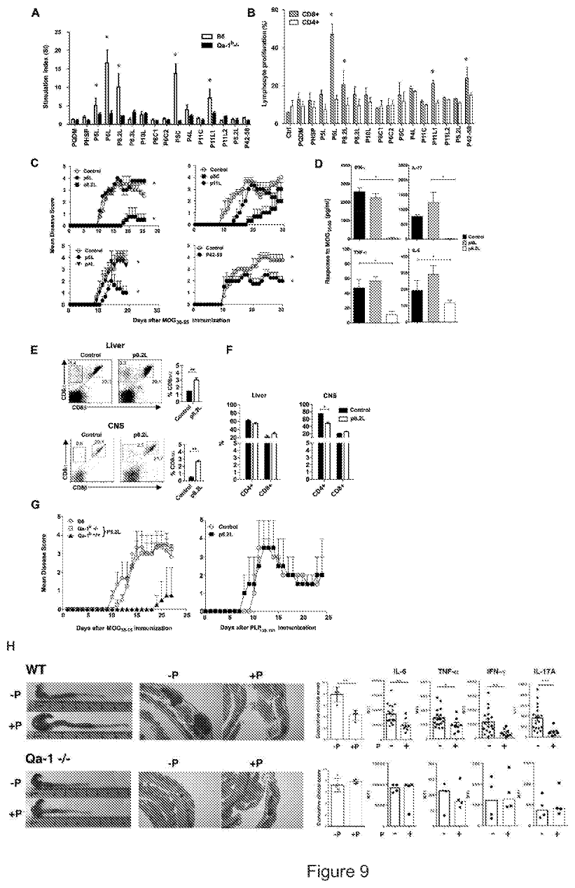

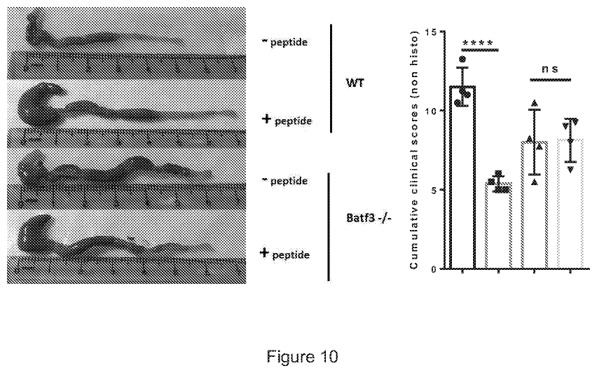

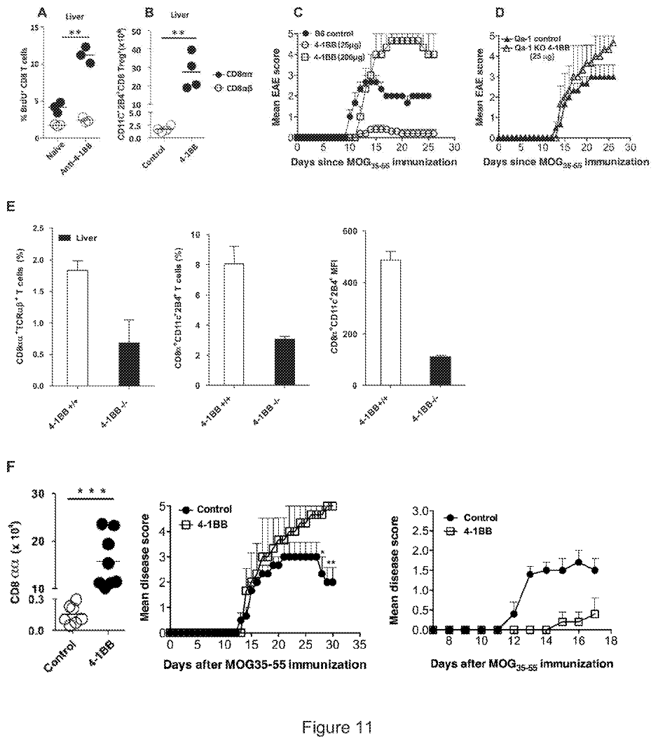

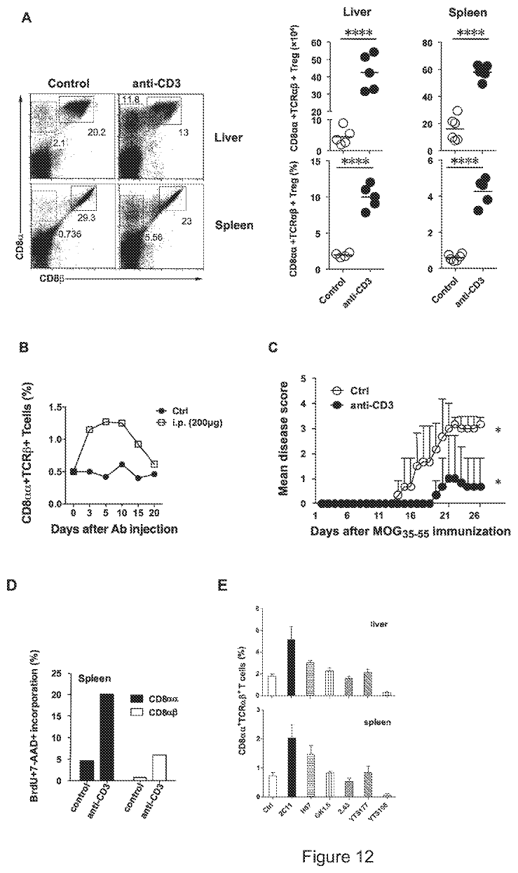

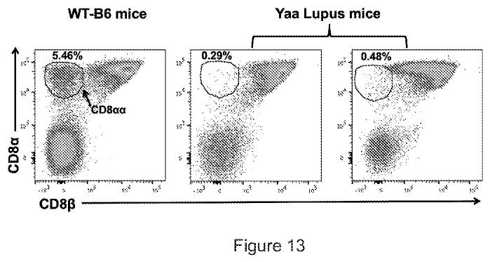

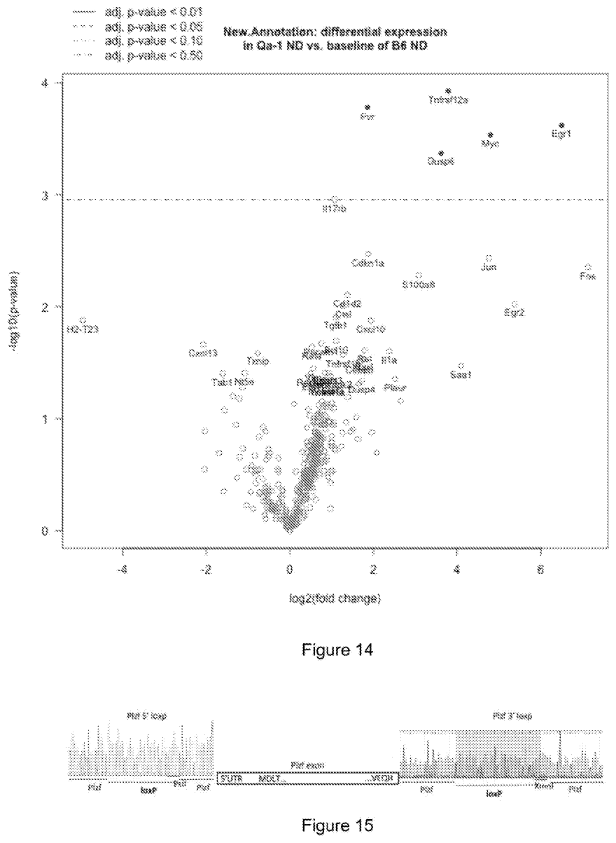

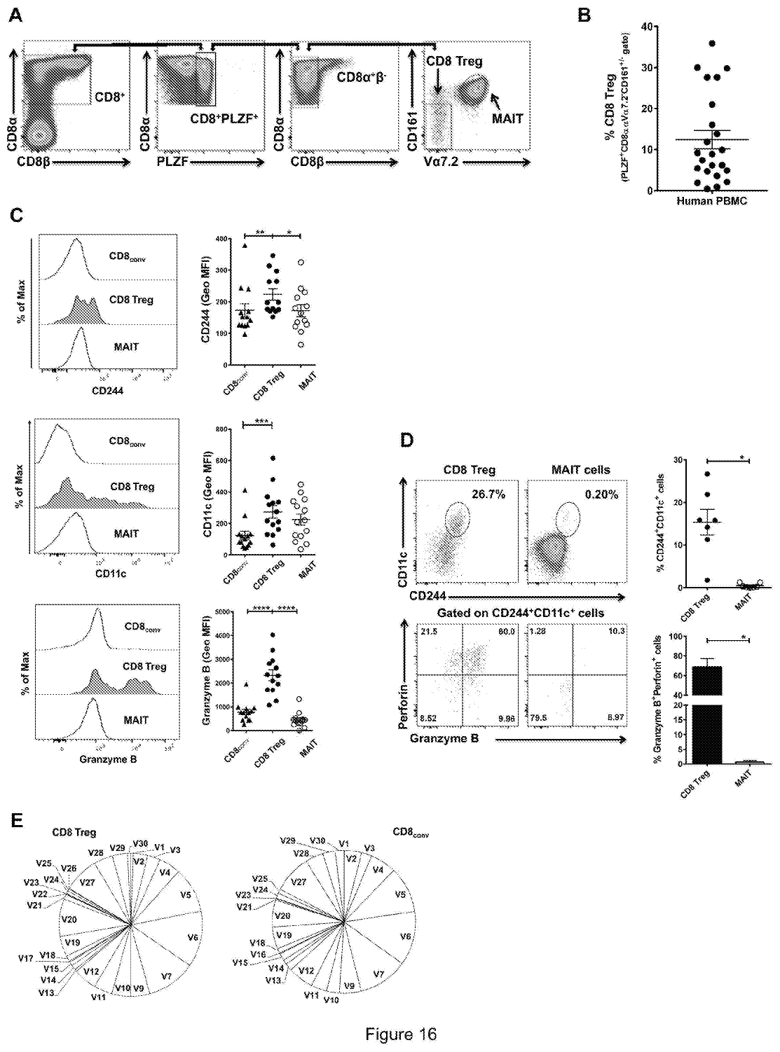

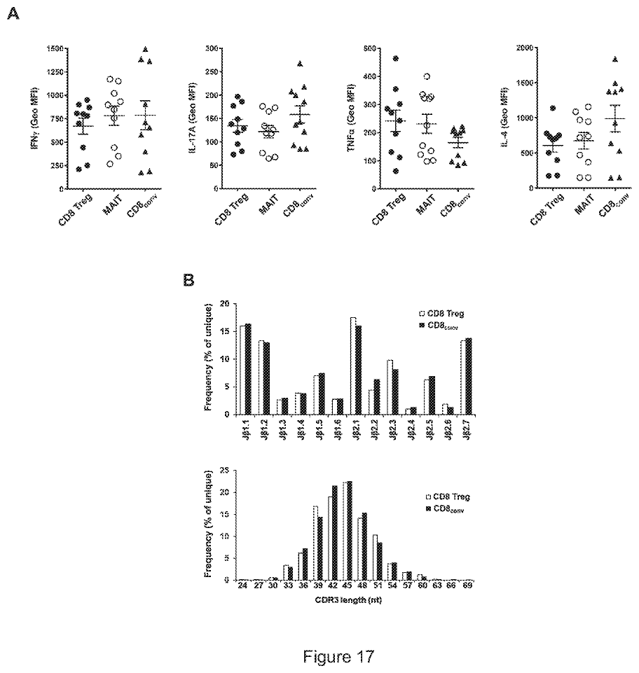

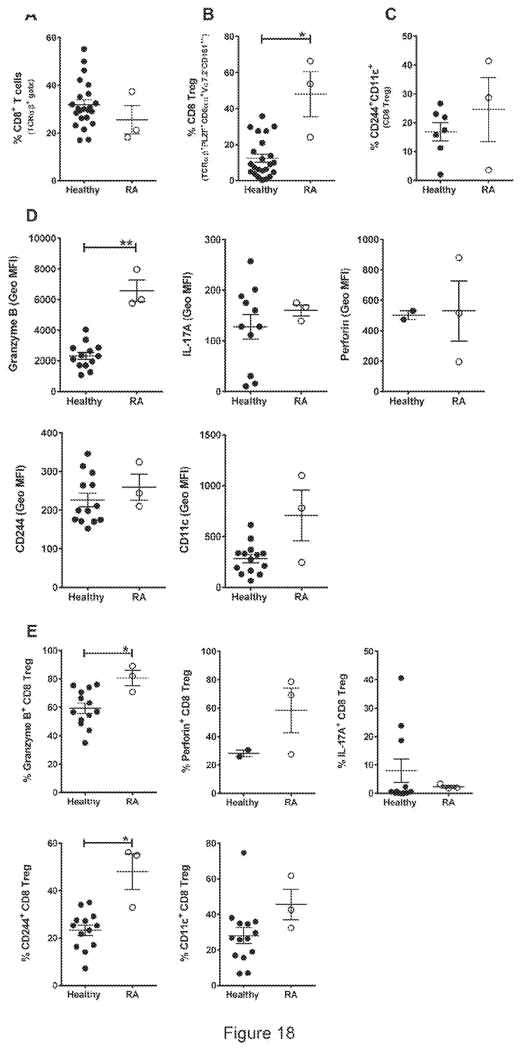

Experimental Strategy: We will use sorted colonic CD8αα Treg to investigate protective efficacy and mechanism using the CD4⁺CD45RB^{high} T cell adoptive transfer model of colitis. The CD4⁺CD45RB^{high} pathogenic T cells will be sorted from naïve B6 mice and co-adoptively transferred into naïve Rag1−/− mice with sorted CD8αα Treg or CD8αβ T cells from colonic tissues. Comparative body weight loss will be monitored following adoptive transfer and mice will be sacrificed to measure colonic inflammation by histopathology and cytokine analysis as shown in FIG. 9. Liver CD8αα Treg will be used as a positive control in these experiments. If colonic CD8αα Treg are able to provide protection in Rag1−/− mice, then in next experiments, colonic CD8αα Treg will be isolated from WT, Perforin−/− and Granzyme B−/− mice and use in adoptive transfer experiments into Rag1−/− mice. Next, we will use sorted colonic CD8αα Treg from PLZF-GFP mice to determine their ability to homing into gut to protect colitis.

To Determine the Role of Qa-1 in CD8αα Treg-Mediated Regulation of CD4 T Cell-Mediated Colitis.

Rationale: Experiments using Qa-1b knockout and knock-in mice or anti-Qa-1 Ab have indicated that the Qa-1 molecules play an important role in the function of CD8 Treg (30-35). Qa-1 in mice (HLA-E in humans), a non-classical MHC class Ib molecule, forms a heterodimer with β2-microglobulin and can present peptides from both self and foreign antigens. Studies in H-2^{u} mice also indicated Qa-1a-dependent apoptotic depletion of only activated but not naïve MBP-reactive Vβ8.2⁺CD4⁺ T cells. Furthermore, Qa-1a-restricted CD8 Treg are expanded in H-2^{u} mice following capture of apoptotic T cells by conventional DCs that mediate cross-presentation of TCRβ-chain-derived peptides to the CD8 Treg. We will determine whether Qa-1 is required for the CD8αα Treg-mediated regulation of colitis.

Experimental Strategy: First, we will determine whether Qa-1 expression on target CD4⁺ T cells is required for CD8αα Treg-mediated regulation of colitis in Rag1−/− mice. To determine this, we will adoptively co-transfer colonic CD8αα Treg from WT mice with CD4⁺CD4.5Rb^{high} T cells from B6 mice or Qa-1b−/− mice into Rag1−/− recipients and monitor colitis. In this case, if Qa-1 expression on target cells is required for suppression, Rag1−/− mice co-transfer with pathogenic CD4⁺ T cells from Qa-1b−/− should develop colitis even in the presence of CD8αα Treg. If we find a Qa-1b-dependent suppression, we will further explore whether there is a Qa-1 allelic difference in regulation. We will use CD8αα Treg from B6 (Qa-1b) or (B6X B6.tla) F1 mice (Qa-1a/b) in these co-transfer experiments. This will clarify whether Qa-1b-restricted CD8αα Treg can suppress Qa-1a⁺ effector cells (Teff) or not,

To further examine whether CD8αα Treg-mediated apoptosis of Teff is cell-cell contact dependent both Teff and CD8αα Treg will be co-cultured in vitro. Sorted CD45.1⁺CD4⁺ Teff and CD45.2⁺ CD8αα Treg will be co-cultured (1:1 to 10:1 ratio) for 72-96 hr in the presence of plate-bound anti-CD3 mAb and the degree of apoptosis (by number of surviving Teff) determined by FACS using staining with PI/Annexin V. To distinguish between anti-proliferative vs. apoptotic mechanisms, we will examine suppression or killing of CFSE-labeled Teff counterstained with PI. It is possible that, in addition to induction of death, the proliferation of PI-negative Teff may also be inhibited indicating that other mechanisms may be involved. We will then use CD8αα Treg from perforin−/− or granzyme B−/− mice to further examine their anti-proliferative or apoptotic role in regulation. Similarly, Teff derived from Qa-1b−/− mice will be used to investigate Qa-1-dependency of immune regulation. Cell contact will be further confirmed using Transwell plates to separate CD8αα Treg and CD4⁺ Teff populations.

To Determine the Role of Qa-1-Restricted CD8αα Treg in Acute and Chronic DSS-Induced Colitis.

Rationale: Our preliminary data show a significant reduction in both colonic and liver CD8αα Treg in Qa-1b−/− mice. Consistent with the role of Qa-1-restricted CD8αα Treg in the maintenance of immune homeostasis in the gut, we found increases secretion of IL-6 and infiltration of CD4⁺ T cells in gut tissue in naïve Qa-1b−/− mice in comparison to naïve WT B6. These data suggest that in the absence of CD8αα Treg, Qa-1−/− mice may be more susceptible to the development of colitis. As mentioned earlier, we have identified several Qa-1-binding peptides derived from the TCR-Vβ chains, which activates splenic and hepatic CD8αα Treg both in vitro and in vivo (data not shown). Here we will use the most potent peptide, p8.2L, that significantly protects mice from DSS-induced colitis in a Qa-1b-dependent manner. Therefore, we will determine the susceptibility of Qa-1b−/− mice to DSS-induced colitis and whether they are protected from colitis following immunization with the Qa-1b-binding peptide p8.2L that induces CD8αα Treg.

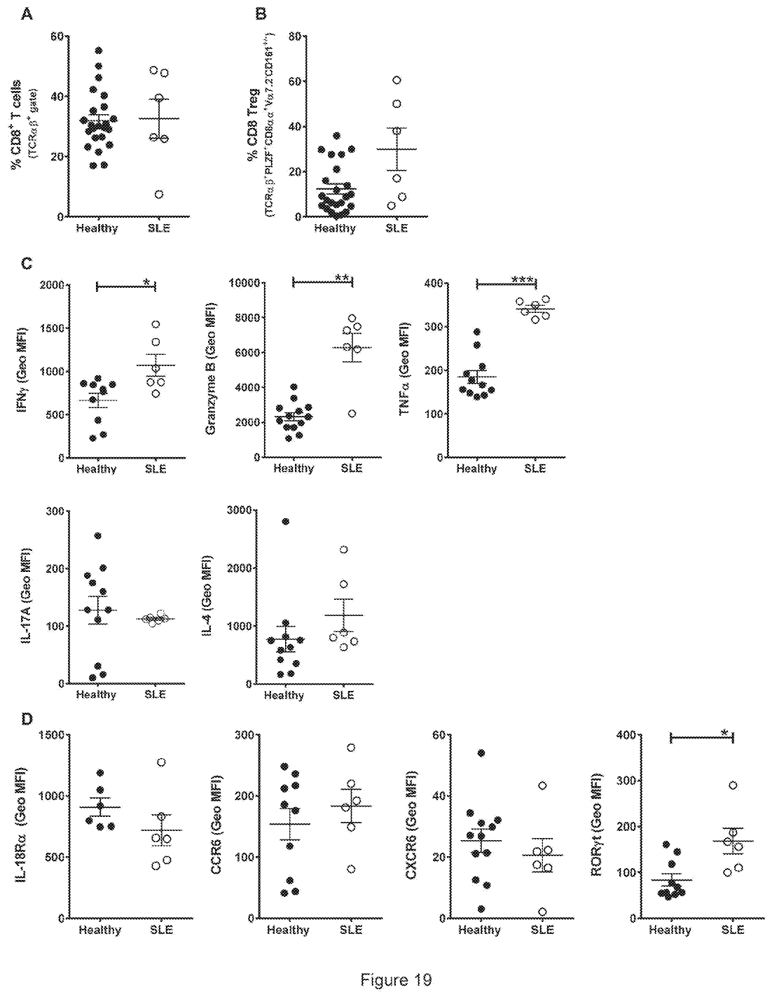

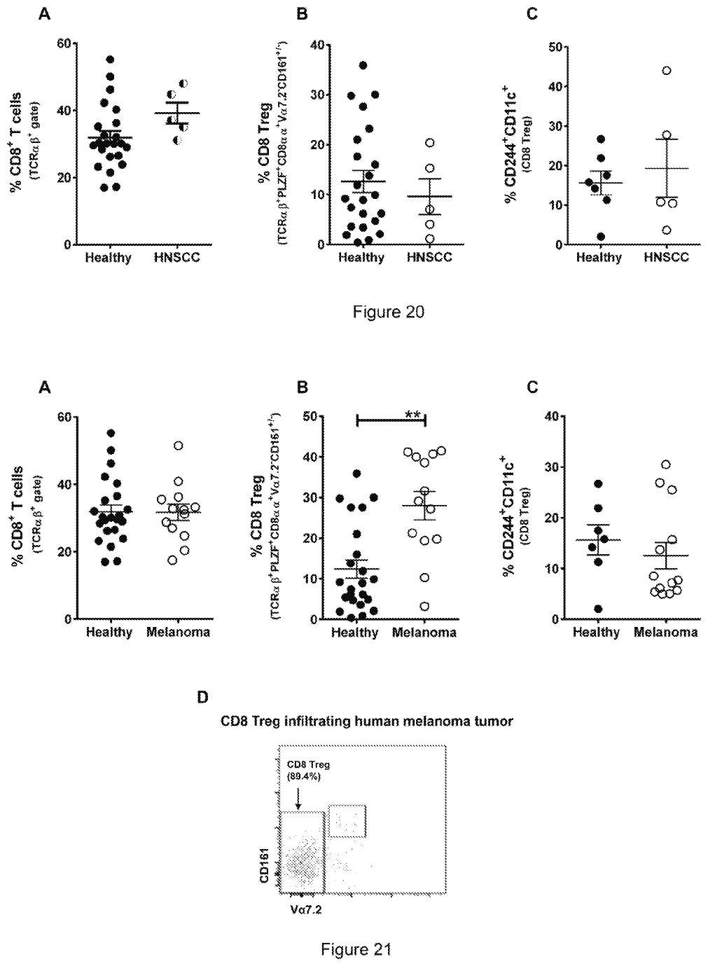

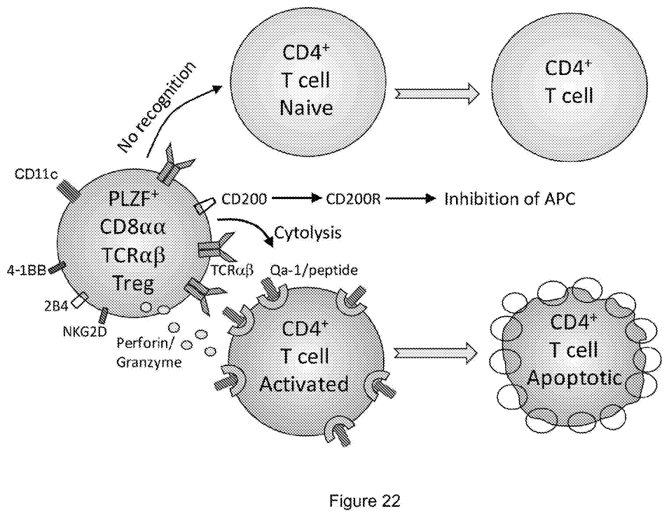

Experimental Strategy: To detect increased sensitivity to colitis in Qa-1−/− mice, WT and Qa-1−/− mice will be exposed to a low dose of DSS (1.5% as opposed to 2.5%) in the acute model and to three doses of 0.5-1% DSS in the chronic model. Comparative colitis will be measured by parameters described in FIG. 19. Next, we will determine whether the Qa-1-binding peptide p8.2L that most potently activates CD8 Treg will be protective both prophylactically and therapeutically in Qa-1−/− mice. To examine efficacy prophylactically, we will administer the peptide p8.2L to WT and Qa-1−/− mice on day 0 and, after 3 days, DSS (2.5%) will be added to drinking water. On the 8th or 9th day following DSS treatment, mice will be sacrificed to measure colitis using parameters as described before. For therapeutic function, we will administer the peptide on day 5 after beginning of DSS treatment when the body weight loss start. Next, we will determine the priming or activation of CD8αα Treg following peptide-immunization by examining CD69 increase and cytokine expression by intracytoplasmic staining. An irrelevant peptide from hen-egg lysozyme will be used as a negative control in these experiments.

Physiological Induction of Colonic CD8αα Treg in the Regulation of Colitis.

Rationale: We will examine the hypothesis that the frequency of colonic CD8αα Treg may differ in different phases of colitis and may correlate with the disease severity. Since CD8αα Treg appear to be involved in the maintenance of homeostasis, we will determine whether the number CD8αα Treg in colon is significantly decreased during the peak of disease and whether it is restored to normal values during the relapsing phase indicating their physiological importance.

Experimental Strategy: We will investigate both acute and chronic models of DSS-induced colitis. In the acute model, we will add 2.5% DSS (Affymetrix) to the feeding water and expose the mice for 7 days. After that, mice will be put on regular water. In the chronic model, mice will be put to 3 cycles of 7 days with 1.5% DSS plus 14 days with regular water. We have verified that 1.5% DSS gives a minimal response in the acute model of the disease. Mice will be sacrificed and colonic (LP and IEL) and MLN tissue will be dissociated into single cell suspensions and analyzed by flow cytometry. For the acute model, days of sacrifice for phenotyping will be 6^{th}, 9^{th}, 16^{th} and 25^{th} and, in the chronic model, will be at the end of each cycle, i.e. days 21^{st}, 42^{nd} and 63^{rd} approximately. Following input in the chronic phase, we will observe mice and sacrifice after every 15 days for phenotyping of CD8αα T cells in colon until the time when body weights are restored. To measure colitis we will use body weight loss, gross colonic features and histopathologic score of colon and cytokine measurement in colon explant culture. In addition, we will use FITC-Dextran tracer to detect epithelial barrier malfunction and Myeloperoxidase (MPO) assay, whenever required.

Potential pitfalls and alternate strategies: There is a possibility that other molecules such as Granzyme A or TRAIL (36)(8)(10) that are not overexpressed in CD8αα Treg in steady state, become involved following activation. We will use other gene deficient mice to explore other pathways. Alternatively, Granzyme B or perforin may have only partial effect, we may have to cross and generate dual perforin/Granzyme B-deficient mice for the isolation of CD8αα Treg and examine their suppressive role. We may alternatively use IL10−/− mice in the C3H/HeJBir background in collaboration with our colleague Dr. Lars Eckmann to determine the role of CD8αα Treg in spontaneous colitis model. Since CD8αα Treg rapidly secrete IL-2, there is a possibility that they may recruit CD4⁺CD25⁺Foxp3⁺ Treg. Attempts will be made to examine this in DSS colitis model following peptide treatment and examining intestinal tissues by FACS on days 2, 4, 8 and 16 for the induction of CD4⁺ Treg and compare to the control peptide-treated mice. If there is paucity of colonic CD8αα Treg for adoptive transfer studies, we may use sorted liver derived CD8αα Treg and then compare their gene signature and other properties. Since DSS colitis is primarily mediated by innate cells, alternatively regulatory mechanism(s) may need to be explored (37)(9)(11).

A detailed knowledge of the cellular and molecular mechanism(s) involved in maintaining immune tolerance in gut is crucial in the development of novel strategies for treatment of IBD. Our central hypothesis is that CD8αα Treg are also enriched in colon and play an important role in limiting the intensity or duration of the inflammatory immune response in gut. This mechanism is different but complimentary from the one mediated by Foxp3⁺CD4⁺ Treg that prevents a damaging response from occurring. Thus, CD8αα Treg-mediated regulation is a negative feedback response in the sense that it allows operation of the natural mechanisms of defense or healing but shuts down these mechanisms after a delay in order to avoid excessive tissue damage (29, 38). Studies proposed here are designed to characterize the colonic PLZF⁺CD8αα Treg and investigate the molecular mechanism(s) involved in the negative feedback immune regulation. Importantly, we have identified Qa-1b-binding peptides that activate CD8αα Treg and significantly protect mice from colitis. Since Qa-1 or HLA-E molecules in human are highly conserved and non-polymorphic, a screen can be setup to identify HLA-E-binding peptides that stimulate human CD8αα Treg with key implications for clinical studies. These studies are highly significant as they will characterize a novel population of colonic CD8αα Treg and will have major implications in designing new strategies for the prevention/and treatment of IBD.

### EXAMPLE 3

A similar phenotype, gene signature and regulatory function of CD8αα Treg in human peripheral blood. We will test the hypothesis that circulating CD8αα Treg also play an important role in the regulation of autoimmunity in humans. Patients with active autoimmune disease may have altered frequency of CD8αα Treg in PBMC and this will be examined after their characterization, including their frequency, cell surface phenotype, cytokine and gene expression profile, MHC-restriction, TCR repertoire and their regulatory function.

It is important to investigate whether the CD8αα Treg equivalent to the mouse counterpart are also present in humans. Our preliminary data using multiparameter flow cytometry analysis suggest that in peripheral blood of healthy individuals, potential CD8αα Treg are present. In human PBMCs, CD8αα Treg are defined as PLZF⁺TCRαβ⁺CD8αα⁺ T cells that do not express the TCRVα7.2/Jα33, which identifies MAIT cells, and have intermediate or very low expression of CD161, different from MAIT cells that are CD1.61^{high}. The frequency of CD8αα Treg was 0.20%±0.05 (mean±sem) that correspond to 2×10³±0.5×10³ in a million of PBMC. Notably, the expression of RORγt, the transcription factor of MAIT cells, as well as CXCR6 and CCR6 in CD8αα Treg was significantly lower than both in MAIT cells and in CD8αβ T cells (data not shown). Similar to murine CD8αα Treg, CD244 or 2B4, and CD11c were also significantly high expressed in human CD8αα Treg. Though IFNγ, IL-17 and IL-4 showed no differences between CD8αα Treg and either CD8αβ T cells or MAIT cells, granzyme B, perforin and TNFα secretion by human CD8αα Treg were significantly increased similar to murine CD8αα Treg (data not shown).

Phenotype analysis: Following isolation of PBMC from EDTA or heparinized blood by Histopaque gradient centrifugation, CD8αα Treg will be identified within the TCRαβ⁺CD8⁺PLZF⁺gate as CD8αα⁺Vα7.2/α33^{neg}CD161^{med/low} and analyzed for the expression of different markers associated with the murine of CD8□□ Treg, including CD122 (IL-2Rβ), CD28, CD127, Foxp3, GITR, CTLA-4, CXCR3, CD45RC, CCR8 and TGF-β, as well as activation markers such as CD25 and CD69. Our preliminary data on murine CD8αα Treg show high expression of CD25, CD28, CD122 and GITR, but not CD127 and FoxP3 in comparison with conventional CD8αβ T cells. However, it is unknown whether human CD8αα Treg also express some of these markers. Also, we will examine the memory status of human CD8αα Tregs based on the expression profile of CCR7 and CD45RA. Previously, it has been described that CD8⁺CD161⁺ and CD8⁺CD122⁺ T cells are memory-like exhibiting a T_{EM} (CCR7⁻CD45RA⁻)/T_{EMRA} (CCR7⁻CD45RA⁺) and T_{CM} (CCR7⁺CD45RA⁻) phenotype, respectively. Fluorescent monoclonal antibodies will be purchased from BD Bioscience, BioLegend or eBioscience.

Cytokine analysis: Our preliminary data indicate that CD8αα Treg in humans also constitutively secrete significantly more granzyme B, TNFα and perforin than MAIT cells and conventional CD8αβ T cells (data not shown), however IFNγ, IL-4 and IL17 secretion was similar between subsets. To investigate the full potential of CD8αα Treg to secrete different cytokines and chemokines, sorted CD8αα Treg and CD8αβ T cells will be stimulated with phorbol 12-myristate 13-acetate (PMA) and ionomycin for 4-6 hours with Monensin (BD Golgi Stop). After stimulation, the frequencies of cytokine-producing cells (IL-2, IFNγ, IL-4, IL-10, IL-13, IL-17, IL-22, TGFβ, TNFα, granzyme B and perforin) will be measured by flow cytometric analysis of intracellular cytokines or by enzyme-linked immunospot (ELISPOT). The culture supernatant will be collected and analyzed using BD™ CBA Human Chemokine Kit: IL-8 (CXCL8/IL-8), RANTES (CCL5/RANTES), monokine induced by interferon-γ (CXCL9/MIG), monocyte chemoattractant protein-1 (CCL-2/MCP-1), and interferon-γ induced protein-10 (CXCL10/IP-10). Alternatively, cytokine gene transcripts will be analyzed by real-time PCR using RNA isolated from sorted. CD8αα Treg and CD8αβ T cells after stimulation with PMA and ionomycin.

Gene expression and the TCR repertoire analysis: To characterize the gene signature of human CD8αα Tregs, we will perform RNA sequencing. (RNA-seq) of freshly sorted CD8αα Treg and conventional CD8αβ T cells from 3 different donors to compare their total RNA expression profile. RNA-seq data of sorted CD8αα Treg and CD8αβ T cells will be generated by an Illumina HiSeq4000 analyzer using the TruSeq v3 Cluster kit at the UCSD Institute for Genomic Medicine as in the case of murine CD8αα Treg. Similar to the analysis of murine CD8□□ Treg, a high throughput sequencing of the TCRβ genes from genomic DNA from sorted CD8αα Treg and CD8αβ T cells will be carried out as described previously (Adaptive Biotechnologies, Seattle, Wash.) (39). The raw sequence data will be preprocessed to remove errors and to compress the data. TCRβ sequences will be analyzed using their ImmunoSEQ Analyzer. For each unique sequence, the nucleotide and predicted amino acid sequence, V (variable). D (diversity) and J (joining) genes and the number of sequencing reads will be determined. The data will be further sorted to exclude any sequence with an out-of-frame rearrangement or a stop codon in the CDR3, and the frequency will be determined for each of the remaining productive unique sequences (nucleotide clonotypes).

Immunoregulatory properties: First, we will examine suppression of T cell proliferation as measured by the dilution of CFSE-labeled (CellTrace™ Thermo Fisher Scientific) sorted autologous CD4⁺CD25⁻ T cells cultured either alone (3×10⁵/well) or with sorted CD8αα Treg (1×10⁵/well) in the presence of anti-CD3/CD28 microbeads. After 72 h of stimulation, cells will be labeled with fluorochrome-conjugated anti-CD8α mAb and intracellular staining with anti-human IFNγ. We predict that CD8αα Treg will be able to suppress both T cell proliferation when co-cultured and the Th1 cytokine secretion profile. To examine MHC restriction, we will add a purified anti-human HLA-E antibody (clone 3D12) to the cultures to block antigen presentation by HLA-E and determine whether the suppressive activity is blocked. Furthermore, we will examine different mechanisms that potentially can be involved in suppression of CD4 T cell proliferation by CD8αα Treg, including neutralizing antibodies against specific soluble factors to reverse inhibition of proliferation and Trans-well experiments to examine whether cell-cell contact is required for apoptosis induction using annexin V staining.

The ability of CD8αα Treg to suppress allogeneic response in a mixed lymphocyte reaction (MLR) is determined. Briefly, PBMCs (responder cells, 1×10⁻⁶ cells/ml) will be stimulated for 6 days in 96-well plates with allogeneic PBMCs previously blocked by mitomycin (blocked, stimulator cells, 0.5×10⁶ cells/ml), ratio 2:1, to perform one-way MLR. After 6 days, cells from the one-way allogeneic MLR will be collected, washed, and CD8αα Treg will be isolated by sorting to evaluate their suppressor properties in a secondary MLR. Thus, freshly isolated PBMCs will be labeled with CFSE (CFSE-labeled, responder cells) and mixed with freshly isolated non-labeled allogeneic PBMCs (stimulator cells) and CD8αα Treg (isolated from the primary one-way MLR), ratio 2:1:0.5. After 4 days, the cells will be collected, washed, stained for CD4 and the proliferation of the CD4⁺ cells will be determined by flow cytometry. We expect that CD8αα Tregs derived from the primary culture will reduce the proliferation of allo-reactive. CD4⁺ T cells in the secondary culture.

Next, we will determine whether CD8αα Treg-mediated suppression involves cytotoxicity to the target cells using a flow cytometry-based cytotoxicity assay that simultaneously measures expression of the degranulation marker CD107a by effector cells (CD8αα Treg) and the apoptosis marker annexin V binding to target cells. Sorted CD4⁺CD25⁻ T cells (Target cells) pulsed with anti-CD³/anti-CD28 beads or non-pulsed, will be labeled with PKH 67 green fluorescent cell linker (Sigma-Aldrich) and mixed with sorted CD8αα Treg (Effector cells) at an E:T ratio of 2:1 to 10:1. The % of CD107a-expressing CD8⁺ T cells and annexin V binding to PKH 67-labeled target cells will be measured by FACS. We anticipate that an increase in both effector cell degranulation and target cell death will be observed only in the presence of CD8αα Tregs and activated target cells. In addition, phenotypic characterization of effector CD8αα Treg will be carried out using different antibodies, including anti-perforin and anti-granzyme B.

### EXAMPLE 4

A Summary of Exemplary Modalities by which CD8αα Treg can be Targeted for Their Activation/Expansion In Vivo and Subsequent Protection from Autoimmune Diseases.

CD8αα Treg can be sorted and adoptive transfer protects from autoimmunity.

A peptide-based modality targets induction of CD8αα Treg which in turns protects from disease.

There is a natural expansion of CD8αα Treg patients with RA and lupus

Anti-4-1bb protects from autoimmunity, e.g., from EAE. Induction of CD8αα Treg following administration of anti-4-1bb antibody and subsequent protection from EAE. CD8αα+TCRαβ+ Treg cells are increased following anti-4-1BB administration. Groups of C57BL/6 mice were either administered with PBS or 4-1BB antibody on day 0. Three days later mice were sacrificed and liver mononuclear cells were isolated and stained with various fluorochrome labeled antibodies to determine the number of CD8αα Treg. EAE is ameliorated in WT B6 mice following anti-4-1BB antibody injection. Groups of female B6 mice (7-8 mice in each) were immunized subcutaneously on day 0 with 100 μg MOG₃₃₋₅₅ peptide emulsified in an equal volume of CFA. On the same day animals were injected intraperitoneally with 25 μg of anti-4-1BB antibody diluted in PBS.

Anti-CD3 protects from autoimmune disease. Induction of CD8αα Treg following administration of anti-CD3 (4c11) antibody and subsequent protection from EAE. CD8αα+TCRαβ+ Treg cells are increased following anti-CD3 administration. Groups of C57BL/6 mice were either administered with PBS or CD3 antibody on day one and day three. On day seven mice were sacrificed and liver mononuclear cells were isolated and stained with various fluorochrome labeled antibodies to detect induction of CD8aa Treg. EAE is ameliorated in WT B6 mice following anti-CD3 injection. Groups of female B6 mice (6 in each) were injected intraperitoneally with 200 μg of anti-CD3 antibody per mouse on day 5. EAE was induced on day 0 by injecting 100 μg MOG₃₃₋₅₅ peptide emulsified in an equal volume of CFA, subcutaneously.

Adoptive transfer of sorted CD8aa Treg protects mice from MOG-induced EAE (a model for multiple sclerosis) and from CD45Rb^{high} CD4+ T cell-induced colitis (a model for IBD) in a perforin-dependent manner.

Administration with CD8+ Treg-inducing peptide protects mice from MOG-induced EAE as well as from DSS-induced colitis in a Qa-1-dependent fashion. Peptide-induced induction of CD8+ Treg protects WT mice but not CD8+Treg-deficient mice from EAE. Peptide-induced induction of CD8+ Treg protects mice from DSS-induced colitis.

Frequency of CD8 Treg is significantly increased in PBMCs derived from patients with ongoing rheumatoid arthritis (RA) and lupus (SLE). Frequency of CD8αα Treg significantly increased in RA patients (n=3) compared to healthy controls (n=26) (*p≤0.05, **p≤0.01, Mann Whitney test).

Circulating CD8αα Treg are increased in SLE patients. The frequency of CD8αα Tregs was significantly increased in PBMC from SLE patients (n=4) compared to healthy controls (n=26) (**p≤0.01, Mann Whitney test).

### EXAMPLE 5

The understanding of immune tolerance is crucial for intervention in autoimmunity and for the generation of an effective anti-tumor immunity. The function of T cells is controlled by both intrinsic (e.g., PD1 and exhaustion) and extrinsic (regulatory T cells or Treg) cell-based mechanisms that prevent them from causing excessive tissue damage. Although earlier studies suggested an important regulatory role for CD8 T cells, a major caveat has hampered their characterization due to lack of molecular markers that differentiate them from conventional non-regulatory CD8 T cells (CD8_{conv}). We have discovered that the expression of PLZF transcription factor in a population of PLZF⁺TCRαβ⁺CD8αα⁺ T cells (hereafter referred as CD8 Treg) in both mice and humans distinguishes them from CD8_{conv}. CD8 Treg are innate-like cells that are enriched in liver of naïve mice and a large proportion of them are CD11c⁺CD244⁺NKG2D⁺NK1.1⁺. Despite having innate-like unconventional T cell features, CD8 Treg are distinct from other innate-like T cells, including mucosal associated invariant T (MAIT) cells (summarized in Table below). Furthermore, their enrichment in liver of naïve mice combined with the PLZF expression and specific cell surface markers differentiate these CD8 Treg from others described earlier by Cantor's laboratory or by ourselves following CFA or TCR-peptide immunizations, respectively (24, 29, 40). The Qa-1-dependence also suggests that CD8 Treg target activated Qa-1⁺ T cells and not naïve Qa-1⁻ T cells, thus, they can control an ongoing T cell response. Accordingly, our preliminary data in indicate that an ongoing autoimmune response may be required for their expansion. Thus, CD8 Treg expansion following autoimmune inflammation and their ability to control activated T cells qualifies them as part of a powerful negative feedback regulatory mechanism that protects tissues from excessive immune-mediated damage. Consistently, we have found a significant increase in the frequency of CD8 Treg in PBMC from lupus and rheumatoid arthritis patients (data not shown) similar to the increase of CD8 Treg during the recovery phase of murine EAE. Studies proposed here are designed to further characterize the biology of CD8 Treg and the molecular mechanism(s) of immune regulation. Furthermore, the expression of 4-1BB on CD8 Treg and the ability of an agonistic anti-4-1BB Ab to expand CD8 Treg and the subsequent protection from EAE suggest that the proposed studies will provide a potentially novel approach to expand/activate CD8 Treg for intervention in humans using the available humanized anti-4-1BB Ab and also offers an explanation for the effects of anti-4-1BB treatment in different experimental conditions. Collectively, these studies are highly significant as they will characterize a novel population of CD8 Treg in both mice and humans with major implications in designing and testing new strategies for the prevention and/or treatment of autoimmune diseases as well as in providing a potential novel checkpoint for effective anti-tumor immunity.

Characteristics distinguishing PLZF⁺CD8⁺ Treg from MAIT cells

| | PLZF⁺CD8 Treg | MAIT cells |
|---|---|---|
| | Mice | |
| TCR repertoire | Polyclonal TCR V□ | Semi-invariant Vα19 |
| | and V□ usage | and limited Vβ |
| Enrichment in B6 | Liver | Lung |
| mice | | |
| Liver phenotype | CD8αα | Double negative and |
| | | CD8αα |
| Transcription | PLZF^{low}, T-bet^{+/-}, | PLZF^{high}, T-bet⁺⁺, |
| factors | Eomes^{+/-} | Eomes⁺⁺ |
| NK1.1 | mostly | ≤20% |
| Surface markers: | CD62L⁺⁺⁺, CD69⁺⁺⁺, | CD62L^{-/+}, CD69^{-/+}, |
| | ICOS⁻, CD103⁻, | ICOS⁺⁺⁺, CD103⁺⁺⁺, |
| | CD127⁻, CD218⁻ | CD127⁺⁺⁺, CD218⁺⁺⁺ |
| Cytokine secretion: | IL-2⁺⁺⁺, IL-17A⁺, | IL-2⁻, IL-17A⁺⁺⁺, |
| | IFNγ⁺, IL-10⁻ | IFNγ⁺, IL-10⁺ |
| Frequency in B | Present (same as WT) | Absent |
| cell-deficient | | |
| (μMT) mice | | |
| Frequency in germ | Present (same as WT) | Absent |
| free mice | | |
| | Human | |
| TCR repertoire | Polyclonal TCR usage | Semi-invariant Vα7.2 |
| | | and limited Vβ |
| PLZF expression | Low | High |
| CD161 expression | Very low to intermediate | High |
| Surface receptors: | IL-18Rα⁺, CXCR6⁺, | IL-18Rα⁺⁺⁺, CXCR6⁺⁺⁺, |
| | CCR6⁺, RORγt⁺ | CCR6⁺⁺⁺, RORγt⁺⁺⁺ |
| Cytokine secretion | Granzyme B⁺⁺⁺ | Granzyme B⁻ |
| MHC-restriction | Qa-1 (mice)/HLA-E (in | MR-1 (both mice and |
| | human) | human) |
| Antigen reactivity | Self-Ags peptides, TCR- | Microbial Ags, |
| | derived peptides | riboflavin |

As proposed above, innate-like unconventional PLZF⁺CD8 Treg are enriched in the liver of naïve mice because the liver provides a more suitable environment for their development (e.g. IL-15-dependency). CD8 Treg with innate-like features offer a rapid mechanism for limiting any excessive immune stimulation to protect tissue against constant exposure to gut-derived antigens. Since this mechanism target only activated but not naïve T cells, it allows effective immunity against microbial antigens to protect the organ.

Phenotype of CD8 Treg is unique and can be distinguished from CD8_{conv}: To our knowledge, we have identified for the first time that PLZF expression in combination with other cell surface markers (PLZF⁺TCRαβ⁺CD8αα⁺) distinguishes CD8 Treg from CD8_{conv} similar to FoxP3expression that differentiates CD4 Treg from conventional CD4⁺ T cells. Furthermore, the presence of PLZF⁺CD8 Treg in germ-free mice, which lack MAIT cells, and in CD1d−/− mice, which lack both NKT and innate-like CD8⁺ T cells, clearly indicate their unique innate phenotype. Also, co-expression of CD11c and CD244 (2B4) on a substantial portion of CD8 Treg further suggests uniqueness of PLZF⁺CD8 Treg in both mice and humans. Additional studies, including Single Cell RNA sequencing analysis, will uncover other cell surface and functional markers for the identification, development and function of CD8 Treg.

Our approach bridges cellular and molecular approaches related to innate T cells Our preliminary data using the fate-mapping strategy (PLZF-Tdtomato expression) indicate that CD8 Treg express PLZF similar to iNKT cells. We now have generated several PLZF-flox founders that will be backcrossed with CD4-Cre mice to generate mice deficient in T cells lacking PLZF to investigate the role of PLZF⁺CD8 Treg in the physiological control of autoimmunity. Identification of this unique population of CD8 Treg (PLZF⁺TCRαβ⁺CD8αα⁺) enriched in liver of naive mice and in healthy humans is quite innovative. In collaboration with Dr. Vijayanand, Single Cell RNA sequencing analysis of CD11c⁺ and CD11c⁻ CD8 Treg in both mouse and human will unravel the transcription program, gene signatures and TCR repertoire related to the regulatory properties. In parallel, the development of multiparameter flow cytometric analysis to identify CD8 Treg in human PBMC is also highly innovative and important. The expression of cell surface molecules, such as CD200, suggests that PLZF⁺CD8 Treg are not only capable of killing target T cells, but they may use negative signaling via CD200 to inhibit the function of APCs, including microglia. We will investigate the role of 4-1BB expression on CD8 Treg and how an agonistic mAb can be used to preferentially activate them with important implications for potential intervention in human autoimmune disease.

Importance of regulatory role for CD8+ T cells, Murine models: Since 1970s the role of CD8⁺ T cells in immune regulation of autoimmune diseases, transplant tolerance and homeostasis of cellular and humoral immune responses has been suggested (8-10), but has not sufficiently advanced as happened for Foxp3⁺ CD4 Treg (14). In mice genetically deficient or depleted of CD8⁺ T cells by treatment with anti-CD8 mAb, an important role for CD8 T cells in regulation of autoimmunity has been shown (11-13). Similarly, a critical regulatory role of CD122⁺CD8⁺ T cells involving a cytolytic mechanism has been shown in IL-2−/− and IL-2Rβ−/− mice (15-17). Interestingly, IL-2/IL-15Rβ-deficiency in humans also leads to a severe combined immunodeficiency syndrome as observed for IL-2Rα (27). Also, CD122⁺CD8⁺ T cells can provide barriers to stem cell engraftment, indicating the clinical relevance of CD8 Treg in humans (41). A critical role for CD8⁺ T cells in IL-2−/− mice was also shown as these animals develop colitis with an accelerated kinetics (18). Human disease: CD8⁺ T cells also have been implicated in various conditions in humans, e.g. transplant survival (19), prevention of inflammatory bowel disease (20) and after treatment of multiple sclerosis with either glatiramer acetate (GA) or vaccination with irradiated, autoreactive CD4⁺ T cells (21, 22). Recently, GA-induced regulatory CD8 T cells have been shown to eliminate CD4⁺ T cells in an HLA-E-restricted manner (21, 23-25). In a clinical trial with anti-CD3 mAb (Teplizumab), increased frequency of memory-like CD8⁺ T cells with regulatory gene expression was found to be associated with a positive clinical response in type 1 diabetes patients (26). Collectively these studies indicate an important role of CD8 T cells in control of autoimmunity.

A novel population of innate-like PLZF⁺CD8 Treg, enriched in liver of naïve mice, can be distinguished from CD8_{conv}. We have identified a population of PLZF⁺TCRαβ⁺CD8αα Treg within the B220⁻CD4⁻ gate expressing only the homodimer CD8αα that represents ˜3.9% (±0.80 SEM) of liver MNCs in naïve B6 mice. PLZF⁺CD8 Treg are also present in bone marrow (˜3.0%), spleen (˜0.5%), blood (0.3%) and lungs (0.3%) of naïve B6 mice as well as in the neonatal thymus (0.5%) until day 5 after birth. In athymic nude mice, which lack all T cells, CD8 Treg were undetectable confirming their thymic origin (data not shown). Since innate-like features in T cells are driven by the expression of PLZF (42-48), we examined its expression in this novel population. CD8 Treg from both B6 and PLZF-eGFP reporter (PEG) mice, which was generated using modified bacterial artificial chromosome transgene expressing eGFP under the control of PLZF regulatory elements (49), express PLZF or GFP, respectively. However, CD8 Treg have lower PLZF expression than iNKT cells (CD4⁺CD1d-αGalCer tetramer⁺). In contrast, PLZF is not expressed by either CD8_{conv} or CD4 T cells. Accordingly, PLZF mRNA expression was found exclusively in sorted CD8 Treg but not in CD8_{conv}. Next, we used the fate-mapping experiment to investigate PLZF expression using PLZF-Cre x R26T mice, in which PLZF-expressing cells are permanently labeled tdTomato (50). Most CD8 Treg in liver of PCre x R26T mice were tdTomato⁺ while CD8_{conv} showed only background level of expression. To further investigate PLZF requirement, we examined the frequency of CD8 Treg in PLZF-deficient mice (PLZF−/−) and heterozygous PLZF+/− and PLZF−/− littermates (49). PLZF−/− mice had significantly reduced numbers of CD8 Treg in liver compared to PLZF+/+ mice. Importantly, these results indicate that CD8 Treg are PLZF⁺ and dependent on its expression.

Adoptive transfer of CD8 Treg protects B6 mice from EAE. CD8 Treg do not express Foxp3 but express glucocorticoid-induced tumor necrosis factor-related receptor (GITR), a marker of active Treg cells (data not shown) (28). To determine the in vivo regulatory potential of CD8 Treg, naïve B6 recipients were adoptively transferred i.v. with 1×10⁵ sorted CD8 Treg or CD8_{conv} isolated from liver of naïve B6 mice before induction of EAE with MOG₃₅₋₅₅/CFA/PTx as described before (51). CD8 Treg but not CD8_{conv} significantly protected mice from EAE. In addition, the significant protection from EAE observed after adoptive transfer of 2×10⁵ sorted CD8 Treg but not CD8_{conv} from liver of CD1d−/− mice eliminated any potential contribution from CD8 NKT cells. These data demonstrate that CD8 Treg have regulatory properties.

A substantial portion of CD8 Treg co-express CD244 and CD11c. The majority of CD8 Treg express CD244 (2B4) (>70%), a marker expressed on NK cells, but also implicated in the regulation of co-stimulation and function of CD8⁺ T cells (52). Notably, CD8 Treg also express CD11c, suggested earlier as a marker on CD8 suppressor T cells (53). Co-staining of CD8 Treg show that all CD11c⁺ CD8 Treg are also CD244⁺ while none of the CD8_{conv} express CD11c or CD244. Next, we examined whether CD244⁺CD11c⁺ CD8 Treg are dependent on PLZF. Both percentage and numbers of CD244⁺CD11c⁺ CD8 Treg were significantly reduced in PLZF−/− mice compared to PLZF+/+ mice. Since CD8 T cells co-expressing CD11c with suppressive functions have been reported (53, 54), we investigated whether CD11c expression on CD8 Treg play a role in their regulatory capacity. Thus, CD8 Treg depleted of CD11c (CD8αα⁺CD11c⁻) (bead-depletion) were adoptively transferred into naïve B6 recipients, and EAE was induced next day. CD11c depletion of CD8 Treg resulted in loss of their ability to control EAE. Consistent with innate-like phenotype, the majority of CD244⁺CD11c⁺ CD8 Treg also express NK1.1 and NKG2D and also express the regulatory molecules CD137 (4-1BB) and CD200 that is absent from CD8_{conv}. Expression of CD137, CD200 and other key immune molecules was also confirmed using RT-PCR (data not shown). Furthermore, CD8 Treg are CD122⁺, displayed a memory/activated phenotype (CD44^{high} CD62L^{low} CD69⁺) and express only NK inhibitory receptors (Ly49A, Ly49E/F, Ly49G2 and Ly49I) but not activating receptors (Ly49D and Ly49H) (data not shown).

MHC-restriction: A significant reduced frequency of CD8 Treg in Qa-1^{b}−/− mice indicate that a large number of CD8 Treg are restricted by Qa-1^{b} molecules. Notably, these CD8 Treg can be distinguish from gut-resident CD8αα⁺TCRαβ⁺ T cells in that they are CD103⁻, PLZF⁺ and are significantly reduced in Qa-1^{b}−/− mice while gut-resident cells are CD103⁺, PLZF⁻ and their frequency do not change in Qa1^{b}−/− mice (data not shown). Furthermore, no changes in their frequency in CD1d−/−, Jα18−/− and μMT−/− suggest that NKT cells and B cells are not required for CD8 Treg, indicating their distinctiveness from NKT-dependent innate-like CD8+ T cells. As expected, while CD8 Treg are absent in CD8α−/− mice, their frequency is increased in CD8β−/− mice. Since Qa-1-restricted T cells can be either TAP-dependent or TAP-independent, the presence of CD8 Treg in TAP1−/− mice suggests that TAP-dependent antigen processing is not required for the development of CD8 Treg. Importantly, CD8 Treg were significantly reduced in both CD122−/− and IL-15−/− mice. Considering that CD122, the β chain receptor for IL-2 and IL-15, is essential for CD8 T cell response to IL-15, these results indicate that IL-15 signaling is necessary for CD8 Treg development.

Cytokine secretion: We have also determined the cytokine secretion profile of sorted CD8 Treg vs. CD8_{conv} after in vitro stimulation with anti-CD3 mAbs. CD8 Treg do not secrete IL-10 or TGFβ, but secrete typical cytokines produced by cytotoxic cells (TNFα, IL-17A) as well as PLZF-driven secretion of both IL-4 and. IFNγ, similar to that in NKT cells (data not shown). It is noteworthy that these cells very rapidly secrete large amounts of IL-2 as well. In the future, we will be investigating whether IL-2 secreted by CD8 Treg may engage Foxp3 Treg that do not secrete IL-2. CD8 Treg also express high levels of perforin and granzyme B (data not shown).

Gene expression profile of murine CD8 Treg using single cell RNA sequencing. We will test the hypothesis that unique characteristics of CD8 Treg, including enrichment in liver, self-reactivity, memory/activated phenotype and innate-like features driven by the PLZF transcription factor, are due to a unique molecular signature. Our preliminary data show that the TCRαβ repertoire of CD8 Treg is polyclonal based on FACS. RT-PCR and high-throughput sequencing (47), indicating their distinctiveness from either MAIT cells that use an invariant TCR Vα19 with biased usage of Vβ8/Vβ6 in mice (42) or NKT cells. However, preliminary data suggest that CD8 Treg are heterogeneous and a substantial portion of them are CD11c⁺. Are CD11c⁺ CD8 Treg also polyclonal? Since the Single Cell RNA (scRNA) sequencing technology allows the simultaneous analysis of the TCRαβ in single cells, we will also examine the TCRαβ repertoire of CD11c⁺ CD8 Treg and compare it with the bulk CD8 Treg population as well as CD11c⁻ CD8 Treg. If the TCR repertoire of CD11c⁺ CD8 Treg is oligoclonal, we plan identify the most common TCR for generation of TCR transgenic or retrogenic mice for their developmental studies as well as for the identification of antigenic peptides recognized by CD8 Treg. Although MAIT cells, which are also PLZF⁺CD8⁺, represent a small population in mice, we will negatively sort out this population using MR1-5-OP-RU tetramers (55) (received from the NIH tetramer facility) for the RNASeq analysis of CD8 Treg similar to our gating strategy in humans.

Single Cell RNA Sequencing Analysis to Identify Gene Signature of CD8 Treg in Mice:

Rationale: We will perform single-cell RNA sequencing of four sorted populations isolated from liver of naïve PLZF-GFP reporter (PEG) mice: PLZF⁻TCRαβ⁺CD8αβ⁺, PLZF⁺TCRαβ⁺CD8αα⁺, PLZF⁺TCRαβ⁺CD8αα⁺CD11c⁺ and PLZF⁺TCRαβ⁺CD8αα⁺CD11c⁻. The gene expression profiles will be compared among these 4 populations for shared genes as well as those that are predominant within each population. Single-cell transcriptome analysis will further help to define new cell-surface markers as well as their molecular profile and the molecular pathways for their identification, development and function.

We plan to perform scRNA sequencing using the Single Cell 3′ Protocol and the 10x™ GemCode™ Technology (56). Thus, liver MNCs from 5-10 naïve PEG mice will be stained and sorted using a FACSAria III into 4 subsets: PLZF⁻TCRαβ⁺CD8αβ⁺, PLZF⁺TCRαβ⁺CD8αα⁺, PLZF⁺TCRαβ⁺CD8αα⁺CD11c⁺ and PLZF⁺TCRαβ⁺CD8αα⁺CD11c⁻. The recommended starting point will be 10,000 sorted cells to generate Gel Bead-In-EMulsions (GEMs). To achieve single cell resolution, the cells are delivered at a limiting dilution, such that the majority (˜90-99%) of generated GEMs contains no cell, while the remainder largely contains a single cell. Approximately, 5,000 cells will be loaded into each Single Cell 3′ Chip. After dissolution of the Single Cell 3′ Gel Bead in a GEM, primers containing (i) an Illumina R1 sequence (read 1 sequencing primer), (ii) a 16 hp 10x Barcode, (iii) a 10 bp randomer and (iv) a poly-dT primer sequence will be released and mixed with cell lysate and Master Mix. The 10x™ GemCode™ Technology will sample a pool of ˜750,000 barcodes to separately index each cell's transcriptome by partitioning thousands of cells into nanoliter-scale Gel Bead-In-EMulsions (GEMs) and will produce full-length, barcoded cDNA that will be amplified by PCR to generate Single Cell 3′ libraries for sequencing and analysis. Single cell RNA sequencing data will be analyzed in collaboration with the Bioinformatics Core at LJI. In addition to the standard analysis, such as demultiplexing, alignment, and gene counting, complementary methods of single-cell differential gene expression (SCDE) and model-based analysis of single-cell transcriptomics (MAST) analysis will be used to compare the full-length transcriptome between the populations. The Benjamini Hochberg test with adjusted P<0.05 and ≥2-fold change will be used to identify differentially expressed genes. Gene set enrichment analysis (GSEA) and ingenuity pathway analysis (IPA) will be also included. We will define shared genes and those differentially expressed either up-regulated or down-regulated between the four populations. The candidate genes will be validated by either real-time PCR with specific primers designed using web-based programs as before (46) or at the protein level using available antibodies for western blotting and/or flow cytometry.

Our preliminary data indicate that CD8 Treg are significantly expanded during EAE in the periphery and that they also infiltrate into the CNS during EAE. It would be important to analyze the TCR repertoire and transcription profile of the CNS-infiltrating CD8 Treg and compare that to CD8_{conv} as well as hepatic CD8 Treg. Since regulatory activity is associated with CD11c expression, it is likely that CD11c⁺ CD8 Treg infiltrate into CNS and negatively signal microglia. Therefore, we will examine and compare the gene expression and TCR repertoire of sorted CD8 Treg and CD8_{conv} isolated from CNS of PEG mice in the recovery phase of EAE. These data should provide additional important information about the nature of CD8 Treg infiltrating the target tissue.

CD11c Expression as a Phenotypic Marker for CD8 Treg:

It is quite interesting that both murine and human CD8 Treg express CD11c. Notably, CD11c⁺CD8⁺T cells have been suggested to be suppressor T cells earlier (53). Several studies on gene expression profiles have suggested that CD11c is also expressed by NK cells, activated T cells, γδT cells and certain macrophage populations (Immunological Genome Project). Also, recently Dr. Jonathan Ashwell's laboratory reported another TCRαβ⁺CD8⁺ T cell subset with DC properties (57). But, our CD8 Treg are distinct from these others in several cell surface markers, including CD44, CD69, CD25, IL-7R, CD122 and PLZF expression. Our preliminary data suggest that CD8 Treg do not express other DC markers, including MHC class II, CD11b, F4/80 and FcRg (data not shown). However, our preliminary data also suggest that around 58% of CD8 Treg express CD11c and that adoptive transfer of CD8⁺ T cells depleted of CD11c⁺ cells resulted in loss of protection from EAE. Since CD11c expression can potentially be used as a phenotypic marker for CD8 Treg in both mice and in humans, it is crucial to address whether CD11c expression on CD8 Treg is regulated by transcriptional mechanisms, which require prior protein synthesis, or acquired through intercellular transfer (58).

We will use the CD11c-EYFP transgenic reporter mice (B6.Cg-Tg(Itgax-Venus)1Mnz/J) in which yellow fluorescent protein (YFP) expression is driven by the CD11c promoter. Considering that high CD11c expression has been shown to be primarily in DC and that only a small portion of cells with intermediate CD11c expression was CD3⁺ in these mice (59), we will characterize the expression of different markers (PLZF, CD244, CD200, NKG2D and 4-1BB) in CD3⁺ cells with intermediate CD11c expression. Therefore, expression of PLZF in CD11c⁺ CD8 Treg in these reporter mice will confirm their transcription regulation rather than their acquisition by intercellular transfer upon chronic activation. It is also clear from our data that CD8 Treg require thymus for development as they are absent in athymic and RAG1−/− mice (data not shown). Also, CD8 Treg express all other markers of classical cytotoxic T cells, including perforin and granzyme B. Thus, a detailed transcriptome RNA sequencing analysis of CD11c⁺ CD8 Treg (above) will further clarify whether a transcription signature of DC lineage or T cell lineage.

PLZF+ CD8 Treg have a specific transcriptional development program and and control control of autoimmunity in a negative feedback regulation. Our hypothesis is that PLZF expression is crucial for the development of CD8 Treg and that chronic autoimmune inflammation ultimately leads to their expansion and physiological control of autoimmunity.

To Determine the Role of PLZF, RORα and Id3 in the Development and Function of CD8 Treg:

It is clear from our preliminary data using antibody staining, GFP reporter mice and fate-mapping strategy that CD8 Treg express PLZF, although their expression levels are lower than iNKT cells. Accordingly, CD8 Treg are significantly reduced in PLZF−/− mice. We will employ bone marrow chimeric mice using PLZF−/− mice as described earlier (49, 60) to further investigate the role of PLZF transcription factor in the development of CD8 Treg. The absence of CD8 Treg in nude mice (data not shown) indicates their thymic origin as well. Our latest preliminary real-time PCR data also suggest enhanced expression of two additional transcription factors, Retinoic acid receptor-related orphan receptor alpha (RORα) and inhibitor-of-DNA-binding (Id)3 (Id3), in CD8 Treg in comparison to CD8_{conv} (data not shown). The transcription factor RORα, is a negative regulator of inflammation (61, 62) while Id3 is essential for the generation of memory CD8⁺ T cells (63). Interestingly, Id2 that is required for the development and functions of innate lymphoid cells (64) and Eomes, which is a critical regulator for CD8⁺ T cell differentiation and effector function (65), are expressed at similar levels in CD8 Treg and CD8_{conv}. Since Id3−/− mice (from Dr. Goldrath at UCSD) and RORα−/− mice (from Jackson Lab) are available, we will investigate their role in the development of CD8 Treg.

Our latest preliminary data indicate a significant loss of CD8 Treg in both RORα−/− and Id3−/− mice similar to that found in PLZF−/− mice. To determine whether the requirement for PLZF, RORα or Id3 is cell-intrinsic to CD8 Treg, irradiated congenic CD45.1 B6 mice will be injected i.v. with bone marrow (BM) cells, depleted of T cells and B cells, isolated from CD45.2 PLZF−/−, RORα−/− or Id3−/− mice and the presence of CD8 Treg monitored by FACS. For comparison, we will also transfer BM cells from wild type littermates into B6 host. If these transcription factors play a cell-intrinsic role, PLZF−/−, RORα−/− or Id3−/− BM transfer may not be able to fully reconstitute B6 host. If this were the case, B6 mice will be reconstituted with a mixture of BM cells isolated from CD45.2 PLZF−/−, RORα−/− or Id3−/− mice and congenic CD45.1 B6 mice at a ratio of 50:50 and the development of CD8 Treg analyzed 12 to 16 weeks post-transfer. These studies should give us important clues regarding the role of these related key transcription factors in the biology of CD8 Treg.

CDS Treg are Primed/Expanded Physiologically to Control Autoimmunity:

Our preliminary data using adoptive transfer of sorted CD8 Treg clearly indicate that CD8 Treg can control autoimmunity. We propose the hypothesis that CD8 Treg are physiologically expanded during the course of EAE and play an important role in the control of disease. Consistent with this hypothesis and the Qa-1^{b} restriction of a large number of CD8 Treg, it has been shown that the recovery as well as susceptibility to re-induction of EAE is compromised in Qa-1−/− mice (30). Notably, our preliminary data suggest that CD8 Treg are expanded physiologically during the recovery phase of EAE. Thus, B6 mice were immunized with either MOG₃₅₋₅₅ for EAE induction as above or an irrelevant peptide derived from hen egg lysozyme (HEL) and hepatic CD8 Treg were analyzed by FACS on day 10 at the onset of disease (EAE d10) or at day 25 during the recovery phase of the disease (EAE d25). CD8 Treg transitorily decreased at the onset of disease (day 10) but significantly expanded during the recovery phase (day 25) of EAE. In contrast, CD8 Treg were not altered in non-diseased mice immunized with an irrelevant HEL peptide. These data indicate that the development of a pro-inflammatory, autoimmune T cell response is important for the expansion of CD8 Treg that are able to control excessive immune response.

Next, we are generating mice that are deficient in PLZF⁺ T cells, including CD8 Treg, using floxed genes in a 2-step process by inserting two loxP sites simultaneously to ensure deletion of the intervening DNA (two double stranded cuts in the DNA followed by repair of the gap by the cell). We have several founders confirmed by sequencing of the entire loci that are being bred now. After crossing with CD4-Cre mice, we will first establish that they are deficient in PLZF⁺ T cells. Based upon our data in PLZF−/− mice, CD4-Cre PLZFflox/flox (PLZF^{F/F}) mice should be deficient in PLZF⁺CD8 Treg cells. Since we are using CD4-Cre mice (CD8-specific Cre is not available), function of NKT cells and a minor population of MAIT cells may also be compromised in these mice. First, groups of PLZF^{F/F} mice and negative littermates will be immunized with PLP₁₇₂₋₁₈₃/CFA/PTX to induce EAE and to be able to address re-induction of disease. Mice will be monitored every day for clinical score and disease onset, severity, incidence and recovery analyzed. Next, we will examine whether susceptibility to re-induction of EAE with PLP₁₇₂₋₁₈₃/CFA is altered in PLZF^{F/F} mice. Thus, PLZF^{F/F} mice and littermates will be immunized with PLP₁₇₂₋₁₈₃/CFA and 30 days later mice will be challenged with PLP/CFA/PTX and disease monitored. We predict that CD8 Treg induced initially in CD8 Treg^{+/+} mice (littermates) will protect them from disease whereas their absence in PLZF^{F/F} mice should allow induction of clinical disease. To rule out the potential caveat that other dysfunctional innate-like T cells in these mice may contribute to the effect, we will adoptively transfer sorted CD8 Treg from negative littermates and determine whether regulation can be reconstituted in PLZF^{F/F} mice. Since CD8 Treg are enriched in liver, we will also investigate whether PLZF^{F/F} mice develop sterile inflammatory liver injury. We will determine whether inflammation and induction of related genes occur in livers of naïve PLZF^{F/F} mice using Nanostring Technology (UCSD Core). These studies will conclusively prove that CD8 Treg are able to not only control physiologically autoimmune disease but also control sterile liver inflammation.

Key cell surface molecules involved in CD8 Treg-mediated immune regulation: Our hypothesis is that CD8 Treg through either cytotoxic mechanism or by engaging other cell surface molecules like CD200 to inhibit activation of APCs, control immune responses. Here, we will determine the role of four different cell surface molecules, 4-1BB, CD244 (2B4), NKG2D and CD200 that are expressed on CD11c⁺CD244⁺ CD8 Treg in both mice and humans.

4-1BB (CD137, tnfrsf9) is a member of the TNF receptor superfamily that is not expressed in naïve T cells but only in activated/memory CD8_{conv} T cells and is involved in their survival and activation (66, 67). Accordingly, agonistic anti-4-1BB Abs injected systemically result in expansion of primarily memory CD8⁺ T cells (68, 69). However, several studies have shown paradoxical outcomes depending upon the timing and doses of anti-4-1BB Abs administration. For example, agonistic anti-4-1BB Ab can enhance some anti-viral and anti-tumor T cell responses while inhibiting CD4⁺ T cell-dependent autoimmunity, including EAE (54, 67). Since 4-1BB is expressed in CD8 Treg in naïve mice, we believe that low dose administration of the agonistic anti-4-1BB in naïve B6 mice leads to expansion/activation of CD8 Treg and subsequent protection from autoimmunity. In contrast, high doses of anti-4-1BB Ab overwhelms the effects on other cells, including activated disease-causing T cells and DCs, and, consequently, the CD8 Treg protective effect is masked. Accordingly, we found that naïve B6 mice injected with a low dose of anti-4-1BB Ab (25 μg/mouse) preferentially expands Brdu⁺ CD8 Treg but not CD8_{conv} and results in a significant protection from EAE. Notably, the protective effect is dependent upon CD8 Treg as B6 mice but not Qa-1−/− mice are protected from EAE. It further indicates that under these conditions CD4 Treg that can also express 4-1BB do not play a significant role. Interestingly, a high dose of anti-4-1BB Ab (200 μg/mouse) does not protect mice from EAE, but rather potentiates disease due to overwhelming activation of encephalitogenic T cells and APCs. Our preliminary data further suggest a significant absence of CD8 Treg (4-1BB⁺CD11c⁺CD244⁺) in 4-1BB−/− mice in comparison to B6 mice (data not shown). Therefore, we will investigate the role of 4-1BB expression on CD8 Treg in the control of EAE and, hopefully, provide an explanation related to its paradoxical effects.

CD244 (also known as 2B4, SLAMF4) is a member of the Ig superfamily and is expressed predominantly on the surface of NK cells and γδ T cells. Unlike the other members of the SLAM family receptors that can engage in homotypic interactions, 2B4 interacts with a affinity receptor SLAMF2 or CD48, which is expressed on a number of hematopoietic cells. The CD244-CD48 interactions can have dual functions either activation or inhibition of the immune response depending upon the degree of receptor expression, extent of ligation and level of adaptor molecules (70-72). An NK-independent regulatory role of CD244 has also been shown in the control of experimental lupus (73). Consistently, a splice variant of CD244 has also been reported to be preferentially expressed in SLE patients and is associated with defective regulation in SLE (74). Since CD244 interactions can control both activation and lysis of target cells, we believe that is important to study the role of CD244 expression in CD8 Treg-mediated immune regulation.

CD200 or OX-2 is a member of Ig superfamily and is expressed in lymphoid cells, including B cells and activated T cells, in both mice and humans. It interacts with its cognate ligand CD200R that is expressed on granulocytes, monocytes, DC and macrophages (75). The interaction of CD200-CD200R inhibits activation of macrophages and microglia and, accordingly, CD200−/− mice develop a rapid and severe form of EAE with enhanced axonal damage (75, 76). Furthermore, CD200-CD200R interactions are also involved in regulation of Th1/Th17 immune responses in arthritis, IBD, transplantation and cancer (77-79). Since CD8 Treg infiltrate into CNS during EAE, we believe that binding of CD200 on CD8 Treg to CD200R expressed on microglia, astrocytes, and oligodendrocytes could suppress axonal damage that is mostly mediated by activated microglia thus protecting mice from EAE.

NKG2D. Similar to NKT cells, CD8 Treg express NK receptors, such as NK1.1 and NKG2D, and these receptors may be involved in fine-tuning CD8 Treg function. Since the in vivo function of NK1.1 is poorly understood, we will focus here on the role of NKG2D in the function of CD8 Treg. NKG2D is encoded by the KLRK1 gene expressed in both mice and humans and binds to cell surface MHC class I-related proteins, MICA/MICB (human) and Rae (mice) (80, 81). In CD8 T cells and NKT cells, NKG2D plays an important role as a co-stimulatory molecule and promotes Th1 cytokine production (82). It can also directly promote cytotoxic killing through excretion of granules containing granzyme B and perforin (83). Consistent with our hypothesis, NKG2D expression is enhanced in inflammatory tissues in autoimmune diseases, including arthritis, lupus and diabetes (80, 81, 84).

We have developed a simple in vitro assay to examine the regulatory activity of CD8 Treg. Specifically, sorted CD8 Treg from B6 or deficient mice will be co-cultured with either CFSE-labeled OVA-reactive OT-II CD4⁺ T cells or sorted CD4⁺ T cells from B6 mice in the presence of OVA peptide or anti-CD3/anti-CD28 beads, respectively. CFSE-dilution and staining with Annexin and PI will be measured by FACS as an indication of response. The conventional CD8αβ T cells will be used as controls. Preliminary data suggest that in addition to the killing of the target CD4⁺ T cells, CD8 Treg appear to inhibit the proliferation of the target CD4⁺ T cells. In some cases, we will use titrated concentration of blocking mAbs, for example anti-CD200 (OX2, Biolegend), in in vitro assays to directly examine effect on regulatory activity. Isotype control antibodies will be used as controls.

CD8 Treg in human also have a similar phenotype, gene signature profile and regulatory function. Our hypothesis is that CD8 Treg are also present in humans and, potentially, play an important role in the regulation of autoimmunity. Consistent with our data showing increased frequency of CD8 Treg only during the recovery phase of ongoing EAE, we have also found an increased frequency of CD8 Treg in PBMCs from arthritis and lupus patients (data not shown).

We have developed a multiparameter flow cytometry analysis to identify CD8 Treg in human PBMCs based on PLZF expression in CD8⁺ T cells and exclusion of Vα7.2/Jα33⁺ CD161^{high} (CD161⁺⁺) MAIT cells (85). Thus. CD8 Treg are PLZF⁺TCRαβ⁺CCD8αα⁺ T cells that express intermediate to very low levels of CD161 (CD161^{+/-}). Following this gating strategy, our preliminary data indicate that CD8 Treg are present in PBL of healthy individuals and represent ˜12% in human PBMC, ranging from 0.4 to 35%. The expression of CD244 and CD11c was significantly higher in human CD8 Treg than in both MAIT and CD8_{conv}. Similar to murine CD8 Treg, a substantial portion of human CD8 Treg also co-express CD244 and CD11c (CD244⁺CD11c⁺) that is almost absent in MAIT cells (˜16% vs ˜1%). Furthermore, the expression of several markers known to be up-regulated in MAIT cells, including IL-18 receptor α (IL-18Rα1), RORγt, CXCR6 and CCR6, was significantly reduced in human CD8 Treg (data not shown). Notably, human CD8 Treg also secreted higher levels of Granzyme B and perforin similar to murine CD8 Treg and also secreted IFNγ, IL-17 and IL-4 (data not shown). In preliminary in vitro suppression assays, sorted human CD8 Treg also inhibits CD4⁺ T cell proliferation significantly (data not shown).

### REFERENCES

1. Crispe I N. 2009. The liver as a lymphoid organ. Annu Rev Immunol 27: 147-63
2. Doherty D G. 2016. Immunity, tolerance and autoimmunity in the liver: A comprehensive review. J Autoimmun 66: 60-75
3. Protzer U, Maini M K, Knolle P A. 2012. Living in the liver: hepatic infections, Nat Rev Immunol 12: 201-13
4. Godfrey D I, Le Nours J, Andrews D M, Uldrich A P, Rossjohn J. 2018. Unconventional T Cell Targets for Cancer Immunotherapy. Immunity 48: 453-73
5. Harriff M J, McMurtrey C, Froyd C A, Jin H, Cansler M, Null M, Worley A, Meemeier E W, Swarbrick G, Nilsen A, Lewinsohn D A, Hildebrand W, Adams E J, Lewinsohn D M. 2018, MR1 displays the microbial metaholome driving selective MR1-restricted T cell receptor usage. Sci Immunol 3
6. Kinjo Y, Illarionov P, Vela J L, Pei B, Girardi E, Li X, Li Y, Imamura M, Kaneko Y, Okawara A, Miyazaki Y, Gomez-Velasco A, Rogers P, Dahesh S, Uchiyama S, Khurana A, Kawahara K, Yesilkaya H, Andrew P W, Wong C H, Kawakami K, Nizet V, Besra G S, Tsuji M, Zajonc D M, Kronenberg M. 2011. Invariant natural killer T cells recognize glycolipids from pathogenic Gram-positive bacteria, Nat Immunol 12: 966-74
7. Ma C, Han M, Heinrich B, Fu Q, Zhang Q, Sandhu M, Agdashian D, Terabe M, Berzofsky J A, Fako V, Ritz T, Longerich T, Theriot C M, McCulloch J A, Roy S, Yuan W, Thovarai V, Sen S K, Ruchirawat M, Korangy F, Wang X W, Trinchieri G, Greten T F. 2018. Gut microbiome-mediated bile acid metabolism regulates liver cancer via NKT cells. Science 360
8. Gershon R K, Kondo K. 1970. Cell interactions in the induction of tolerance: the role of thymic lymphocytes. Immunology 18: 723-37
9. Vlad G, Cortesini R, Suciu-Foca N. 2005. License to heal: bidirectional interaction of antigen-specific regulatory T cells and tolerogenic APC. J Immunol 174: 5907-14
10. Kumar V. 2004. Homeostatic control of immunity by TCR peptide-specific Tregs. J Clin Invest 114: 1222-6
11. Jiang H, Zhang S I, Pemis B. 1992. Role of CD8+ T cells in murine experimental allergic encephalomyelitis. Science 256: 1213-5
12. Koh D-R, Fung-Leung W-P, Ho A, Gray D, Acha-Orbea H, T. W. M. 1992. Less mortality but more relapses in experimental allergic encephalomyeltis in CD8−/− mice. Science 256: 1210-3
13. Tabi Z, McCombe P A, Pender M P. 1994. Apoptotic elimination of V beta 8.2+ cells from the central nervous system during recovery from experimental autoimmune encephalomyelitis induced by the passive transfer of V beta 8.2+ encephalitogenic T cells. Eur J Immunol 24: 2609-17
14. Sakaguchi S. 2004. Naturally arising CD4+ regulatory t cells for immunologic self-tolerance and negative control of immune responses. Annu Rev Immunol 22: 531-62
15. Rifa'i M, Kawamoto Y, Nakashima I, Suzuki H. 2004. Essential roles of CD8+CD122+ regulatory T cells in the maintenance of T cell homeostasis. J Exp Med 200: 1123-34
16. Suzuki H, Kundig T M, Furlonger C, Wakeham A, Timms E, Matsuyama T, Schmits R, Simard J J, Ohashi P S, Griesser H, et al. 1995. Deregulated T cell activation and autoimmunity in mice lacking interleukin-2 receptor beta. Science 268: 1472-6
17. Suzuki H, Shi Z, Okuno Y, Isobe K. 2008. Are CD8+CD122+ cells regulatory T cells or memory T cells? Hum Immunol 69: 751-4
18. Simpson S J, Mizoguchi E, Allen D, Bhan A K, Terhorst C. 1995. Evidence that CD4+, but not CD8+ T cells are responsible for murine interleukin-2-deficient colitis. Eur J Immunol 25: 2618-25
19. Colovai A I, Mirza M, Vlad G, Wang S, Ho E, Cortesini R. Suciu-Foca N. 2003. Regulatory CD8+CD28− T cells in heart transplant recipients. Hum Immunol 64: 31-7
20. Brimnes J, Allez M, Dotan I, Shao L, Nakazawa A, Mayer L. 2005. Defects in CD8+ regulatory T cells in the lamina propria of patients with inflammatory bowel disease. J Immunol 174: 5814-22
21. Karandikar N J, Crawford M P, Yan X, Ratts R B, Brenchley J M, Ambrozak D R, Lovett-Racke A E, Frohman E M, Stastny P, Douek D C, Koup R A, Racke M K. 2002. Glatiramer acetate (Copaxone) therapy induces CD8(+) T cell responses in patients with multiple sclerosis. J Clin Invest 109: 641-9
22. Zhang J, Medaer R, Stinissen P, Hafler D, Raus J. 1993. MHC-restricted depletion of human myelin basic protein-reactive T cells by T cell vaccination. Science 261: 1451-4
23. Correale J, Villa. A. 2008, Isolation and characterization of CD8+ regulatory T cells in multiple sclerosis. J Neuroimmunol 195: 121-34
24. Sinha S, Itani F R, Karandikar N J. 2014. Immune regulation of multiple sclerosis by CD8+ T cells. Immunol Res 59: 254-65
25. Tennakoon D K, Mehta R S, Ortega S B, Bhoj V, Racke M K, Karandikar N J. 2006. Therapeutic induction of regulatory, cytotoxic CD8+ T cells in multiple sclerosis. J Immunol 176: 7119-29
26. Tooley J E, Vudattu N, Choi J, Cotsapas C. Devine L, Raddassi K, Ehlers M R, McNamara J G, Harris K M, Kanaparthi S, Phippard D, Herold K C. 2016. Changes in T-cell subsets identify responders to FcR-nonbinding anti-CD3 mAb (teplizumab) in patients with type 1 diabetes. Eur J Immunol 46: 230-41
27. Gilmour K C, Fujii H, Cranston T, Davies E G, Kinnon C, Gaspar H B. 2001. Defective expression of the interleukin-2/interleukin-15 receptor beta subunit leads to a natural killer cell-deficient form of severe combined immunodeficiency. Blood 98: 877-9
28. Ronchetti S, Ricci E, Petrillo M G, Cari L, Migliorati G, Nocentini G, Riccardi C. 2015. Glucocorticoid-induced tumour necrosis factor receptor-related protein: a key marker of functional regulatory T cells. J Immunol Res 2015: 171520
29. Smith T R, Kumar V. 2008. Revival of CD8(+) Treg-mediated suppression. Trends Immunol
30. Hu D, Ikizawa K, Lu L, Sanchirico M E, Shinohara M L, Cantor H. 2004. Analysis of regulatory CD8 T cells in Qa-1-deficient mice. Nat Immunol 5: 516-23
31. Kim H J, Cantor H. 2011. Regulation of self-tolerance by Qa-1-restricted CD8(+) regulatory T cells. Semin Imnunol 23: 446-52
32. Kim H J, Verbinnen B, Tang X, Lu L, Cantor H. 2010. Inhibition of follicular T-helper cells by CD8(+) regulatory T cells is essential for self tolerance. Nature 467: 328-32
33. Tang X, Maricic I, Kumar V. 2007. Anti-TCR antibody treatment activates a novel population of nonintestinal CD8 alpha alpha+ TCR alpha beta+ regulatory T cells and prevents experimental autoimmune encephalomyelitis. J Immunol 178: 6043-50
34. Tang X, Maricic I, Purohit N, Bakamjian B, Reed-Loisel L M, Beeston T, Jensen P, Kutnar V. 2006. Regulation of immunity by a novel population of Qa-1-restricted CD8alphaalpha+TCRalphabeta+ T cells. J Immunol 177: 7645-55
35. Tang X L, Smith T R, Kumar V. 2005. Specific control of immunity by regulatory CD8 T cells. Cell Mol Immunol 2: 11-9
36. Ren X, Ye F, Jiang Z, Chu Y, Xiong S, Wang Y. 2007, Involvement of cellular death in TRAIL/DR5-dependent suppression induced by CD4(+)CD25(+) regulatory T cells. Cell Death Differ 14: 2076-84
37. Schippers A, Muschaweck M, Clahsen T, Tautorat S, Grieb L, Tenbrock K, Gassier N, Wagner N. 2016. beta7-Integrin exacerbates experimental DSS-induced colitis in mice by directing inflammatory monocytes into the colon. Mucosal Immunol 9: 527-38
38. Germain R N. 2012. Maintaining system homeostasis: the third law of Newtonian immunology. Nat Immunol 13: 902-6
39. Marrero I, Aguilera C, Hamm D E, Quinn A, Kumar V. 2016. High-throughput sequencing reveals restricted TCR Vbeta usage and public TCRbeta clonotypes among pancreatic lymph node memory CD4(+) T cells and their involvement in autoimmune diabetes. Mol Immunol 74; 82-95
40. Jiang H, Chess L. 2000. The specific regulation of immune responses by CD8+ T cells restricted by the MHC class Ib molecule, Qa-1. Annu Rev Immunol 18: 185-216
41. MacKenzie-Graham A J, Pribyl T M, Kim S, Porter V R, Campagnoni A T, Voskuhl R R. 1997. Myelin protein expression is increased in lymph nodes of mice with relapsing experimental autoimmune encephalomyelitis. Immunol 159: 4602-10
42. Eidson M, Wahlstrom J, Beaulieu A M, Zaidi B, Carsons S E, Crow P K, Yuan J, Wolchok J D, Horsthemke B, Wieczorek D, Sant'Angelo D B. 2011. Altered development of NKT cells, gammadelta T cells, CD8 T cells and NK cells in a PLZF deficient patient. PLoS One 6: e24441
43. Fergusson J R, Huhn M H, Swadling L, Walker L J, Kurioka A, Llibre A, Bertoletti A, Hollander G, Newell E W, Davis M M, Sverremark-Ekstrom E, Powrie F, Capone S, Folgori A. Barnes E, Willberg C B, Ussher J E, Klenerman P. 2016. CD161(int)CD8+ T cells: a novel population of highly functional, memory CD8+ T cells enriched within the gut. Mucosal Immunol 9: 401-13
44. Fergusson J R, Smith K E, Fleming V M, Rajoriya N, Newell E W, Simmons R, Marchi E, Bjorkander S, Kang Y H, Swadling L, Kurioka A, Sahgal N, Lockstone H, Baban D, Freeman G J, Sverremark-Ekstrom E, Davis M M, Davenport M P, Venturi V, Ussher J E, Willberg C B, Klenerman P. 2014. CD161 defines a transcriptional and functional phenotype across distinct human T cell lineages, Cell Rep 9; 1075-88
45. Gordon S M, Carty S A, Kim J S, Zou T, Smith-Garvin J, Alonzo E S, Haimm E, Sant'Angelo D B, Koretzky G A, Reiner S L, Jordan M S. 2011. Requirements for eomesodermin and promyelocytic leukemia zinc finger in the development of innate-like CD8+ T cells. J Immunol 186: 4573-8
46. Marrero I, Ware R, Kumar V. 2015. Type II NKT Cells in Inflammation, Autoimmunity, Microbial Immunity, and Cancer. Front Immunol 6: 316
47. Savage A K, Constantinides M G, Flan J, Picard D, Martin E, Li B, Lantz O, Bendelac A. 2008. The transcription factor PLZF directs the effector program of the NKT cell lineage. Immunity 29: 391-403
48. Weinreich M A, Odumade O A, Jameson S C, Hogquist K A. 2010. T cells expressing the transcription factor PLZF regulate the development of memory-like CD8+ T cells. Nat Immunol 11: 709-16
49. Kovalovsky D, Uche O U, Eladad S, Hobbs R M, Yi W, Alonzo E, Chua K, Eidson M, Kim H J, Im J S, Pandolfi P P, Sant'Angelo D B. 2008. The BIB-zinc finger transcriptional regulator PLZF controls the development of invariant natural killer cell effector functions. Nat Immunol 9: 1055-64
50. Zhang S, Laouar A, Denzin L K, Sant'Angelo D B. 2015. Zbtb16 (PLZF) is stably suppressed and not inducible in non-innate T cells via T cell receptor-mediated signaling. Sci Rep 5: 12113
51. Maricic I, Halder R, Bischof F, Kumar V. 2014. Dendritic cells and anergic type I NKT cells play a crucial role in sulfatide-mediated immune regulation in experimental autoimmune encephalomyelitis. J Immunol 193: 1035-46
52. Assarsson E, Kambayashi T, Persson C M, Chambers B J, Ljunggren H G. 2005. 2B4/CD48-mediated regulation of lymphocyte activation and function. J Immunol 175: 2045-9
53. Vinay D S, Kwon B S. 2010. CD11c+CD8+ T cells: two-faced adaptive immune regulators. Cell Immunol 264: 18-22
54. Vinay D S, Kwon B S. 2016. Therapeutic potential of anti-CD137 (4-1BB) monoclonal antibodies. Expert Opin Ther Targets 20: 361-73
55. Rahimpour A, Koay H F, Enders A, Clanchy R, Eckle S B, Meehan B, Chen Z, Whittle B, Liu L, Fairlie D P, Goodnow C C, McCluskey J, Rossjohn J, Uldrich A P, Pellicci D G, Godfrey D I. 2015. Identification of phenotypically and functionally heterogeneous mouse mucosal-associated invariant T cells using MRI tetramers. J Exp Med 212: 1095-108
56. Patil V S, Madrigal A, Schmiedel B J, Clarke J, O'Rourke P, de Silva A D, Harris E, Peters B, Seumois G, Weiskopf D, Sette A, Vijayanand P. 2018. Precursors of human CD4(+) cytotoxic T lymphocytes identified by single-cell transcriptome analysis. Sci Immunol 3
57. Kuka M, Munitic I, Ashwell J D. 2012. Identification and characterization of polyclonal alphabeta-T cells with dendritic cell properties. Nat Commun 3: 1223
58. Rechavi O, Goldstein I, Kloog Y. 2009. Intercellular exchange of proteins: the immune cell habit of sharing, FEBS Lett 583: 1792-9
59. Lindquist R L, Shakhar G, Dudziak D, Wardemann H, Eisenreich T, Dustin M L, Nussenzweig M C. 2004. Visualizing dendritic cell networks in vivo. Nat Immunol 5: 1243-50
60. Lynch L, Michelet X, Zhang S, Brennan P J, Moseman A, Lester C, Besra G, Vomhof-Dekrey E E, Tighe M, Koay H F, Godfrey D I, Leadbetter E A, Sant'Angelo D B, von Andrian U, Brenner M B. 2015. Regulatory iNKT cells lack expression of the transcription factor PLZF and control the homeostasis of T(reg) cells and macrophages in adipose tissue. Nat Immunol 16: 85-95
61. Dzhagalov I, Giguere V, He Y W. 2004. Lymphocyte development and function in the absence of retinoic acid-related orphan receptor alpha. J Immunol 173: 2952-9
62. Leppkes M, Becker C, Ivanov, II, Hirth S, Wirtz S, Neufert C, Pouly S, Murphy A J, Valenzuela D M, Yancopoulos G D, Becher B, Littman D R, Neurath M F. 2009. RORgamma-expressing Th17 cells induce murine chronic intestinal inflammation via redundant effects of IL-17A and IL-17F. Gastroenterology 136: 257-67
63. Ji Y, Pos Z, Rao M, Klebanoff C A, Yu Z, Sukumar M, Reger R N, Palmer D C, Borman Z A, Muranski P, Wang E, Schrump D S, Marincola F M, Restifo N P, Gattinoni L. 2011. Repression of the DNA-binding inhibitor Id3 by Blimp-1 limits the formation of memory CD8+ T cells. Nat Immunol 12: 1230-7
64. Mjosberg J. Bernink J, Peters C, Spits H. 2012. Transcriptional control of innate lymphoid cells. Eur J Immunol 42: 1916-23
65. Pearce E L, Mullen A C, Martins G A, Krawczyk C M, Hutchins A S, Zediak V P, Banica M, DiCioccio C B, Gross D A, Mao C A, Shen H, Cereb N, Yang S Y, Lindsten T, Rossant J, Hunter C A, Reiner S L. 2003. Control of effector CD8+ T cell function by the transcription factor Eomesodermin. Science 302: 1041-3
66. Croft M, Siegel R M. 2017. Beyond TNF: TNF superfamily cytokines as targets for the treatment of rheumatic diseases. Nat Rev Rheumatol 13: 217-33
67. Wang C, Lin G H, McPherson A J, Watts T H. 2009. Immune regulation by 4-1BB and 4-19BL: complexities and challenges, Immunol Rev 229: 192-215
68. Shuford W W, Klussman K, Tritchler D D, Loo D T, Chalupny J. Siadak A W, Brown T J, Emswiler J, Raecho H, Larsen C P, Pearson T C, Ledbetter J A, Aruffo A, Mittler R S. 1997. 4-1BB costimulatory signals preferentially induce CDS+ T cell proliferation and lead to the amplification in vivo of cytotoxic T cell responses. J Exp Med 186: 47-55
69. Takahashi C, Mittler R S, Vella A T. 1999. Cutting edge: 4-1BB is a bona fide CD8 T cell survival signal. J immunol 162: 5037-40
70. Chlewicki L K, Velikovsky C A, Balakrishnan V, Mariuzza R A, Kumar V. 2008. Molecular basis of the dual functions of 2B4 (CD244). J Immunol 180: 8159-67
71. McArdel S L, Terhorst C, Sharpe A H. 2016. Roles of CD48 in regulating immunity and tolerance. Clin Immunol 164: 10-20
72. Waggoner S N, Kumar V. 2012. Evolving role of 2B4/CD244 in T and NK cell responses during virus infection. Front Immunol 3: 377
73. Brown D R, Calpe S, Keszei M, Wang N, McArdel S, Terhorst C, Sharpe A H. 2011. Cutting edge: an NK cell-independent role for Slamf4 in controlling humoral autoimmunity. J Immunol 187: 21-5
74. Kis-Toth K, Comte D, Karampetsou M P, Kyttaris V C, Kannan L, Terhorst C, Tsokos G C, 2016. Selective Loss of Signaling Lymphocytic Activation Molecule Family Member 4-Positive CD8+ T Cells Contributes to the Decreased Cytotoxic Cell Activity in Systemic Lupus Erythematosus. Arthritis Rheumatol 68: 164-73
75. Barclay A N, Wright G J, Brooke G, Brown M H. 2002. CD200 and membrane protein interactions in the control of myeloid cells. Trends Immunol 23: 285-90
76. Hoek R , Ruuls S R, Murphy C A, Wright G J, Goddard R, Zurawski S M, Blom B, Homola M E, Streit W J, Brown M H, Barclay A N, Sedgwick J D. 2000. Down-regulation of the macrophage lineage through interaction with OX2 (CD200). Science 290: 1768-71
77. Elshal M F, Aldahlawi A M, Saadah O I, McCoy J P. 2015. Reduced Dendritic Cells Expressing CD200R1 in Children with Inflammatory Bowel Disease: Correlation with Th17 and Regulatory T Cells. Int J Mol Sci 16: 28998-9010
78. Gao S, Hao B, Yang X F, Chen W Q. 2014. Decreased CD200R expression on monocyte-derived macrophages correlates with Th17/Treg imbalance and disease activity in rheumatoid arthritis patients. Inflamm Res 63: 441-50
79. Liu J Q, Talebian F, Wu L, Liu Z, Li M S, Wu L, Zhu J, Markowitz J, Carson W E, 3rd, Basu S, Bai X F. 2016. A Critical Role for CD200R Signaling in Limiting the Growth and Metastasis of CD200+ Melanoma. J Immunol 197: 1489-97
80. Joshi S K, Lang M L. 2013. Fine tuning a well-oiled machine: Influence of NK1.1 and NKG2D on NKT cell development and function. Int Immunopharmacol 17: 260-6
81. Lanier L L. 2015. NKG2D Receptor and Its Ligands in Host Defense. Cancer Immunol Res 3; 575-82
82. Jelencic V, Lenartic M, Wensveen F M, Polic B. 2017. NKG2D: A versatile player in the immune system. Immunol Lett
83. Kuylenstierna C, Bjorkstrom N K, Andersson S K, Sahlstrom P, Bosnjak L, Paquin-Proulx D, Malmberg K J, Ljunggren H G, Moll M, Sandberg J K. 2011. NKG2D performs two functions in invariant NKT cells: direct TCR-independent activation of NK-like cytolysis and co-stimulation of activation by CD1d. Eur J Immunol 41: 1913-23
84. Guerra N, Pestal K, Juarez T, Beck J, Tkach K, Wang L, Runlet D H. 2013. A selective role of NKG2D in inflammatory and autoimmune diseases. Clin Immunol 149: 432-9
85. Porcelli S, Yockey C E, Brenner M B, Balk S P. 1993. Analysis of T cell antigen receptor (TCR) expression by human peripheral blood CD4-8-alpha/beta T cells demonstrates preferential use of several V beta genes and an invariant TCR alpha chain. J Exp Med 178: 1-16

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to identify or detect immune cells having TCRαβ+, CD8αα+, Nk1.1+, PLZF+, CD161+, and optionally having one or more of CD11c+, CD137+ CD244+, or one or more of NK-inhibitory receptors, comprising contacting a sample having mammalian immune cells with a ligand that binds CD8αα, a ligand that binds NK1.1, a ligand that binds PLZF, and a ligand that binds CD161, and optionally a ligand that binds CD11c, a ligand that binds CD137, a ligand that binds CD244, a ligand that binds TCRαβ, or a ligand that binds NK-inhibitory receptors; and identifying or detecting an amount of a population of cells CD8αα+, Nk1.1+, PLZF+, and CD161+, and optionally having one or more of CD11c+, CD137+ CD244+ TCRαβ+, or one or more of NK-inhibitory receptors.

2. The method of claim 1 wherein the cells are identified using antibodies specific for TCRαβ, CD8αα, NK1.1, PLZF, or CD161, and optionally antibodies specific for one or more of CD11c, CD137, CD244, or one or more of NK-inhibitory receptors.

3. The method of claim 1 wherein the cells are human cells.

4. The method of claim 1 further comprising isolating the identified cells.

5. The method of claim 4 further comprising expanding the isolated cells.

6. The method of claim 5 wherein the cells are from a patient with an autoimmune disease.

7. The method of claim 4 wherein the cells are cultured with IL-2, IL-15, Qa-1/HLA-E binding peptides, anti-CD3 antibodies or anti-CD137 antibodies, or any combination thereof.

8. A method to decrease the number of immune cells having TCRαβ, CD8αα, Nk1.1, PLZF, and CD161, and optionally having one or more of CD11c, CD137, CD244, or one or more of NK-inhibitory receptors in a mammal, comprising: administering to the mammal an effective amount of a composition comprising one or more antibodies specific for CD8αα, specific for Nk1.1, specific for PLZF, or specific for CD161, or a combination thereof, and optionally a composition comprising one or more antibodies specific for CD11c, specific for CD137, specific for CD244, specific for TCRαβ, or one or more of NK-inhibitory receptors, or a combination thereof or administering to the mammal an effective amount of one or more antibodies specific for CD8αα, specific for Nk1.1, specific for PLZF or specific for CD161, or a combination thereof, and optionally one or more antibodies specific for CD11 c, specific for CD137, specific for CD244, specific for TCRαβ, or one or more of NK-inhibitory receptors, or a combination thereof.

9. (canceled)

10. A method to prevent, inhibit or treat cancer in a mammal, comprising: administering to the mammal an effective amount of a composition comprising one or more antibodies specific for CD8αα, specific for Nk1.1, specific for PLZF, or specific for CD161, or a combination thereof, and optionally a composition comprising one or more antibodies specific for CD11c, specific for CD137, specific for CD244, specific for TCRαβ, or specific for one or more of NK-inhibitory receptors, or a combination thereof, or administering to the mammal an effective amount of one or more antibodies specific for CD8αα, specific for Nk1.1, specific for PLZF, or specific for CD161, or a combination thereof, and optionally one or more antibodies specific for CD11c, specific for CD137, specific for CD244, specific for TCRαβ, or specific for one or more of NK-inhibitory receptors, or a combination thereof.

11. (canceled)

12. The method of claim 10 wherein the cancer is neck cancer.

13. The method of claim 10 wherein the cancer is melanoma.

14. The method of claim 10 wherein the cancer is head cancer.

15. A method to prevent, inhibit or treat autoimmune disease in a mammal, comprising: administering to the mammal a composition comprising one or more Qa-1/HLA-E binding peptides, anti-CD11c antibodies, anti-CD3 antibodies or anti-CD137 antibodies, or any combination thereof, in an amount effective to stimulate CD8a+ T regs or administering to the mammal one or more Qa-1/HLA-E binding peptides, anti-CD11c antibodies, anti-CD3 antibodies or anti-CD137 antibodies, or any combination thereof, in an amount effective to stimulate CD8a+T regs.

16-17. (canceled)

18. The method of claim 15 wherein the one or more antibodithat are specific for CD11c and CD137, CD11c and CD3, or CD3 and CD137.

19. The method of claim 15 wherein the disease is IBD, colitis, lupus or RA or autoimmune liver disease.

20-21. (canceled)

22. The method of claim 15 wherein the T or B cells in the mammal with the disease are increased relative to a mammal without the disease.

23. A method to prevent, inhibit or treat organ or graft rejection in a mammal, comprising: administering to the mammal a composition comprising one or more Qa-1/HLA-E binding peptides, anti-CD11c antibodies, anti-CD3 antibodies or anti-CD137 antibodies, or any combination thereof, in an amount effective to stimulate CD8a+T regs or administering to the mammal one or more Qa-1/HLA-E binding peptides, anti-CD11c antibodies, anti-CD3 antibodies or anti-CD137 antibodies, or any combination thereof, in an amount effective to stimulate CD8a+T regs.

24. (canceled)

25. The method of claim 23 wherein the mammal is a human.

26. The method of claim 10 wherein the peptide(s), antibodies, or a combination thereof, or the composition, is systemically administered.

27. The method of claim 10 wherein the peptide(s), antibodies, or a combination thereof, or the composition, is locally administered.
